(19) United States
(12) Patent Application Publication
(10) Pub. No.: US 2024/0108737 A1
(43) Pub. Date: Apr. 4, 2024

(54) ANTI-INFECTIVE BICYCLIC PEPTIDE LIGANDS

(71) Applicant: BicycleTx Limited, Cambridge (GB)

(72) Inventors: Nicholas KEEN, Carlisle, MA (US); Gemma MUDD, Cambridge (GB); Katerine VAN RIETSCHOTEN, Cambridge (GB); Katie GAYNOR, Cambridge (GB); Liuhong CHEN, Cambridge (GB); Maximilian HARMAN, Cambridge (GB); Michael SKYNNER, Cambridge (GB); Paul BESWICK, Cambridge (GB); Mehdi AMOURA, Cambridge (GB); Simone GIORGI, Cambridge (GB); Giulia LATTANZI, Cambridge (GB); Iain LINGARD, Cambridge (GB)

(21) Appl. No.: 18/271,333

(22) PCT Filed: Jan. 10, 2022
(86) PCT No.: PCT/GB2022/050036

§ 371 Date: Jul. 7, 2023

Related U.S. Application Data
(60) Provisional application No. 63/135,321, filed on Jan. 8, 2021.

(51) Int. Cl. A61K 47/64; A61P 31/14

(52) U.S. Cl. CPC A61K 47/64; A61P 31/14

(57) ABSTRACT

The present invention relates to multimers of polypeptides which are covalently bound to molecular scaffolds such that two or more peptide loops are subtended between attachment points to the scaffold. The invention also describes the multimerization of polypeptides through various chemical linkers and hinges of various lengths and rigidity using different sites of attachments within polypeptides. In particular, the invention describes multimers of peptides which are high affinity binders of severe acute respiratory syndrome coronavirus 2 (SARS-CoV-2), particularly the spike protein S1 of SARS-CoV-2. The invention also includes pharmaceutical compositions comprising said polypeptides and to the use of said polypeptides in suppressing or treating a disease or disorder mediated by infection of SARS-CoV-2 or for providing prophylaxis to a subject at risk of infection of SARS-CoV-2.

### FIELD OF THE INVENTION

The present invention relates to multimers of polypeptides which are covalently bound to molecular scaffolds such that two or more peptide loops are subtended between attachment points to the scaffold. The invention also describes the multimerization of polypeptides through various chemical linkers and hinges of various lengths and rigidity using different sites of attachments within polypeptides. In particular, the invention describes multimers of peptides which are high affinity binders of severe acute respiratory syndrome coronavirus 2 (SARS-CoV-2), particularly the spike protein S1 of SARS-CoV-2. The invention also includes pharmaceutical compositions comprising said polypeptides and to the use of said polypeptides in suppressing or treating a disease or disorder mediated by infection of SARS-CoV-2 or for providing prophylaxis to a subject at risk of infection of SARS-CoV-2.

### BACKGROUND OF THE INVENTION

Coronavirus disease 2019 (COVID-19) is an infectious disease caused by severe acute respiratory syndrome coronavirus 2 (SARS-CoV-2). The disease was first identified in December 2019 in Wuhan, the capital of China's Hubei province, and spread globally, resulting in a pandemic. Common symptoms include fever, cough, and shortness of breath. Other symptoms may include fatigue, muscle pain, diarrhea, sore throat, loss of smell, and abdominal pain. The time from exposure to onset of symptoms is typically around five days but may range from two to fourteen days. While the majority of cases result in mild symptoms, some progress to viral pneumonia and multi-organ failure. As of 6 Jan. 2021, more than 86 million cases have been reported globally, resulting in more than 1.8 million deaths.

The virus is primarily spread between people during close contact, often via droplets produced by coughing, sneezing, or talking. While these droplets are produced when breathing out, they usually fall to the ground or onto surfaces rather than being infectious over long distances. People may also become infected by touching a contaminated surface and then their face. The virus can survive on surfaces for up to 72 hours. It is most contagious during the first three days after the onset of symptoms, although spread may be possible before symptoms appear and in later stages of the disease.

Currently, there is no vaccine or specific antiviral treatment for COVID-19. Management involves treatment of symptoms, supportive care, isolation, and experimental measures. The World Health Organization (WHO) declared the 2019-2020 coronavirus outbreak a Public Health Emergency of International Concern (PHEIC) on 30 Jan. 2020 and a pandemic on 11 Mar. 2020. Local transmission of the disease has been recorded in many countries across all six WHO regions.

There is therefore a great need to provide an effective prophylactic and/or therapeutic treatment intended to avoid or ameliorate the symptoms associated with infection of SARS-CoV-2, such as COVID-19.

### SUMMARY OF THE INVENTION

According to a first aspect of the invention, there is provided a multimeric binding complex which comprises at least two differing bicyclic peptide ligands, each of which comprises a peptide ligand specific for severe acute respiratory syndrome coronavirus 2 (SARS-CoV-2) comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

According to a further aspect of the invention, there is provided a pharmaceutical composition comprising the multimeric binding complex as defined herein in combination with one or more pharmaceutically acceptable excipients.

According to a further aspect of the invention, there is provided the multimeric binding complex as defined herein for use in suppressing or treating a disease or disorder mediated by infection of SARS-CoV-2 or for providing prophylaxis to a subject at risk of infection of SARS-CoV-2.

### BRIEF DESCRIPTION OF THE FIGURES

FIG. 1: Hamster in vivo SARS-CoV-2 Challenge Study with BCY18656.

### DETAILED DESCRIPTION OF THE INVENTION

According to a first aspect of the invention, there is provided a multimeric binding complex which comprises at least two differing bicyclic peptide ligands, each of which comprises a peptide ligand specific for severe acute respiratory syndrome coronavirus 2 (SARS-CoV-2) comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

The present invention describes a series of multimerized bicyclic peptides with various chemical linkers and hinges of various lengths and rigidity using different sites of attachments within said bicyclic peptide which bind and activate SARS-CoV-2 with a wide range of potency and efficacy.

It will be appreciated by the skilled person that the concept of the invention is the recognition that multiply arranged (multimeric) bicyclic peptides provide a synergistic benefit by virtue of the resultant properties of said multimeric binding complexes compared to the corresponding monomeric binding complexes which contain a single bicyclic peptide. For example, the multimeric binding complexes of the invention typically have greater levels of binding potency or avidity (as measured herein by Kd values) than their monomeric counterparts. Furthermore, the multimeric binding complexes of the invention are designed to be sufficiently small enough to be cleared by the kidneys.

Without being bound by theory it is believed that multimerized bicyclic peptides are able to activate receptors by hetero-crosslinking more than one target within SARS-CoV-2. Thus, the multimeric binding complex comprises at least two differing bicyclic peptide ligands. By “differing” it is meant bicyclic peptides having a different amino acid sequence. In this embodiment, the differing bicyclic peptide ligands within the multimeric binding complex will bind to different epitopes on SARS-CoV-2—the resultant target bound complex will therefore create a biparatopic (if the multimeric complex comprises two differing bicyclic peptides), triparatopic (if the multimeric complex comprises three differing bicyclic peptides) or tetraparatopic (if the multimeric complex comprises four differing bicyclic peptides), etc.

Without being bound by theory it is believed that multimerized bicyclic peptides are able to activate receptors by hetero-crosslinking differing targets, such as differing target receptors on SARS-CoV-2. Thus, in one embodiment, said bicyclic peptide ligands are specific for different targets on SARS-CoV-2. It will be appreciated that in this embodiment, the multimeric binding complex comprises at least two differing bicyclic peptide ligands (i.e. bicyclic peptide ligands having differing amino acid sequences). In this embodiment, each of the bicyclic peptides within the multimeric binding complex will bind a differing epitope upon SARS-CoV-2—the resultant target bound complex will therefore create a bispecific multimeric binding complex (if the multimeric complex comprises two differing bicyclic peptides), trispecific multimeric binding complex (if the multimeric complex comprises three differing bicyclic peptides), tetraspecific multimeric binding complex (if the multimeric complex comprises four differing bicyclic peptides), etc.

It will be appreciated that the multimeric binding complexes of the invention may be designed to be capable of binding to a range of different targets on SARS-CoV-2, such as receptors.

The bicyclic peptides within the multimeric binding complexes of the invention may be assembled via a number of differing options. For example, there may be a central hinge or branching moiety with spacer or arm elements radiating from said hinge or branch point each of which will contain a bicyclic peptide. Alternatively, it could be envisaged that a circular support member may hold a number of inwardly or outwardly projecting bicyclic peptides.

In one embodiment, each bicyclic peptide ligand is connected to a central hinge moiety by a spacer group.

It will be appreciated that the spacer group may be linear and connect a single bicyclic peptide with the central hinge moiety. Thus, in one embodiment, the multimeric binding complex comprises a compound of formula (I):

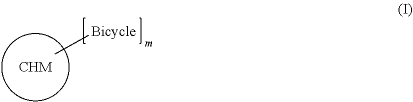

wherein CHM represents a central hinge moiety;
Bicycle represents a bicyclic peptide ligand as defined herein; and
m represents an integer selected from 2 to 10.

In one embodiment, m represents an integer selected from 3 to 10. In a further embodiment, m represents an integer selected from 2, 3 or 4.

In a further embodiment, m represents 2.

When m represents 2, it will be appreciated that the central hinge moiety will require 2 points of attachment. Thus, in one embodiment, m represents 2 and CHM is a motif of formula (A):

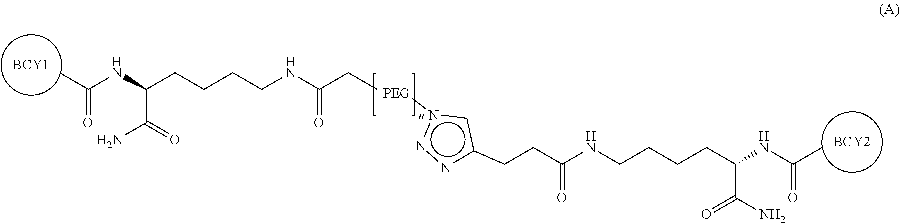

wherein BCY1 and BCY2 represent differing bicyclic peptide ligands.

### Bicyclic Peptide Ligands

It will be appreciated that the multimeric binding complexes herein will comprise a plurality of monomeric bicyclic peptides specific for SARS-CoV-2.

### SARS-CoV-2 Bicyclic Peptide Monomers

In one embodiment, said peptide ligand is specific for the spike protein of SARS-CoV-2. The spike protein (S protein) is a large type I transmembrane protein of SARS-CoV-2. This protein is highly glycosylated as it contains 21 to 35 N-glycosylation sites. Spike proteins assemble into trimers on the virion surface to form the distinctive “corona”, or crown-like appearance. The ectodomain of all CoV spike proteins share the same organization in two domains: a N-terminal domain named S1 that is responsible for receptor binding and a C-terminal S2 domain responsible for fusion. CoV diversity is reflected in the variable spike proteins (S proteins), which have evolved into forms differing in their receptor interactions and their response to various environmental triggers of virus-cell membrane fusion.

In a further embodiment, said peptide ligand binds to either the S1 of S2 domain of the spike protein (S protein). In a yet further embodiment, said peptide ligand binds to the S1 domain of the spike protein (S1 protein). Without being bound by theory it is believed that binding to the S1 domain of SARS-CoV-2, namely the receptor binding domain of SARS-CoV-2, will prevent the virus from binding to its target (thought to be ACE2 bound to the surface of lung airway cells) to enter tissue and cause disease.

In one embodiment, said loop sequences comprise 2, 3, 4, 5, 6, 7 or 8 amino acids.

In one embodiment, said loop sequences comprise three reactive groups separated by two loop sequences one of which consists of 3 amino acids and the other of which consists of 6 amino acids.

In a further embodiment, said loop sequences comprise three reactive groups separated by two loop sequences one of which consists of 3 amino acids and the other of which consists of amino acids and the bicyclic peptide ligand comprises an amino acid sequence which is selected from:

(SEQ ID NO: 1)
CᵢHHACᵢᵢPILTGWCᵢᵢᵢ;
(SEQ ID NO: 6)
CᵢPHACᵢᵢPSLWGWCᵢᵢᵢ;
(SEQ ID NO: 7)
CᵢLHACᵢᵢPRLTHWCᵢᵢᵢ;
(SEQ ID NO: 8)
CᵢLHACᵢᵢQYLWGYCᵢᵢᵢ;
(SEQ ID NO: 9)
CᵢSHACᵢᵢPRLFGWCᵢᵢᵢ;
(SEQ ID NO: 10)
CᵢQHACᵢᵢPYLWDYCᵢᵢᵢ;
(SEQ ID NO: 58)
CᵢPFACᵢᵢHKLYGWCᵢᵢᵢ;
(SEQ ID NO: 59)
CᵢMKACᵢᵢPYLYGWCᵢᵢᵢ;
(SEQ ID NO: 60)
CᵢRHACᵢᵢTHLYGHCᵢᵢᵢ;
(SEQ ID NO: 61)
CᵢPYACᵢᵢTRLYGWCᵢᵢᵢ;
(SEQ ID NO: 62)
CᵢSHACᵢᵢPRLTGWCᵢᵢᵢ;
(SEQ ID NO: 63)
CᵢLHSCᵢᵢPRLSGWCᵢᵢᵢ;
(SEQ ID NO: 64)
CᵢRHSCᵢᵢPILTGWCᵢᵢᵢ;
(SEQ ID NO: 65)
CᵢGHSCᵢᵢPVLWGWCᵢᵢᵢ;
(SEQ ID NO: 66)
CᵢPHSCᵢᵢPKLFGWCᵢᵢᵢ;
and
(SEQ ID NO: 67)
CᵢTHSCᵢᵢPYLFGWCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 3 amino acids and the other of which consists of 6 amino acids, the molecular scaffold is TATA and the bicyclic peptide ligand additionally comprises N- and/or C-terminal additions and comprises an amino acid sequence which is selected from:
A-(SEQ ID NO: 1)-A (herein referred to as BCY15230);
A-(SEQ ID NO: 6)-A (herein referred to as BCY15235);
A-(SEQ ID NO: 7)-A (herein referred to as BCY15236);
A-(SEQ ID NO: 8)-A (herein referred to as BCY15237);
A-(SEQ ID NO: 9)-A (herein referred to as BCY15238);
A-(SEQ ID NO: 10)-A (herein referred to as BCY15239);
A-(SEQ ID NO: 58)-A (herein referred to as BCY15364);
A-(SEQ ID NO: 59)-A (herein referred to as BCY15365);
A-(SEQ ID NO: 60)-A (herein referred to as BCY15366);
A-(SEQ ID NO: 61)-A (herein referred to as BCY15367);
A-(SEQ ID NO: 62)-A (herein referred to as BCY15368);
A-(SEQ ID NO: 63)-A (herein referred to as BCY15369);
A-(SEQ ID NO: 64)-A (herein referred to as BCY15370);
A-(SEQ ID NO: 65)-A (herein referred to as BCY15371);
A-(SEQ ID NO: 66)-A (herein referred to as BCY15372); and
A-(SEQ ID NO: 67)-A (herein referred to as BCY15373).

In a still yet further embodiment, said loop sequences comprise three reactive groups separated by two loop sequences one of which consists of 3 amino acids and the other of which consists of 6 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 6)-A-[Sar_{6]}-[KFl] (herein referred to as BCY15303); and
A-(SEQ ID NO: 63)-A-[Sar_{6]}-[KFl] (herein referred to as BCY15329).

In an alternative embodiment, said loop sequences comprise three reactive groups separated by two loop sequences one of which consists of 4 amino acids and the other of which consists of 6 amino acids.

In a further embodiment, said loop sequences comprise three reactive groups separated by two loop sequences one of which consists of 4 amino acids and the other of which consists of 6 amino acids and the bicyclic peptide ligand comprises an amino acid sequence which is selected from:

(SEQ ID NO: 29)
CᵢLTNDCᵢᵢHSDIRYCᵢᵢᵢ;
and
(SEQ ID NO: 30)
CᵢITNDCᵢᵢHTSLIFCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 4 amino acids and the other of which consists of 6 amino acids, the molecular scaffold is TBMT and the bicyclic peptide ligand additionally comprises N- and/or C-terminal additions and comprises an amino acid sequence which is selected from:
A-(SEQ ID NO: 29)-A (herein referred to as BCY15335); and
A-(SEQ ID NO: 30)-A (herein referred to as BCY15336).

In a still yet further embodiment, said loop sequences comprise three reactive groups separated by two loop sequences one of which consists of 4 amino acids and the other of which consists of 6 amino acids, the molecular scaffold is TBMT, the bicyclic peptide additionally comprises N- and/or C-terminal additions and a labelling moiety, such as fluorescein (Fl), and comprises an amino acid sequence which is:
A-(SEQ ID NO: 30)-A-[Sar₆]-[KFl] (herein referred to as BCY15314).

In an alternative embodiment, said loop sequences comprise three reactive groups separated by two loop sequences one of which consists of 4 amino acids and the other of which consists of 8 amino acids.

In a further embodiment, said loop sequences comprise three reactive groups separated by two loop sequences one of which consists of 4 amino acids and the other of which consists of 8 amino acids and the bicyclic peptide ligand comprises an amino acid sequence which is selected from:

(SEQ ID NO: 3)
CᵢVDANCᵢᵢKIKILQRMCᵢᵢᵢ;
(SEQ ID NO: 4)
CᵢTSSVCᵢᵢKIKELQRKCᵢᵢᵢ;
(SEQ ID NO: 5)
CᵢRSLLCᵢᵢEYLQRTDSCᵢᵢᵢ;
(SEQ ID NO: 14)
CᵢLTKSCᵢᵢKIKMLQRVCᵢᵢᵢ;
(SEQ ID NO: 15)
CᵢMQPSCᵢᵢRVLQLQRVCᵢᵢᵢ;
(SEQ ID NO: 16)
CᵢALPSCᵢᵢRILHLQHRCᵢᵢᵢ;
(SEQ ID NO: 17)
CᵢHDAHCᵢᵢKILELQHRCᵢᵢᵢ;
(SEQ ID NO: 18)
CᵢTSSHCᵢᵢRVLEEQRLCᵢᵢᵢ;
(SEQ ID NO: 19)
CᵢPRDRCᵢᵢPTAWLYGLCᵢᵢᵢ;
(SEQ ID NO: 20)
CᵢAEAGCᵢᵢRVKQLQQICᵢᵢᵢ;
(SEQ ID NO: 21)
CᵢTPSPCᵢᵢRVKELQRACᵢᵢᵢ;
(SEQ ID NO: 26)
CᵢSTANCᵢᵢRILELQQLCᵢᵢᵢ;
(SEQ ID NO: 44)
CᵢVGRLCᵢᵢSTATDIRKCᵢᵢᵢ;
(SEQ ID NO: 48;
herein referred to as BCY16983
when complexed with TATB)
CᵢRQSQCᵢᵢDWWAIRSFCᵢᵢᵢ;
(SEQ ID NO: 49)
CᵢTDATCᵢᵢSIKRLQRLCᵢᵢᵢ;
(SEQ ID NO: 50)
CᵢSPVSCᵢᵢPSGFKFGLCᵢᵢᵢ;
(SEQ ID NO: 68)
CᵢDSPWCᵢᵢRIRSLQRQCᵢᵢᵢ;
(SEQ ID NO: 69)
CᵢSVGACᵢᵢRVKLLQRVCᵢᵢᵢ;
and
(SEQ ID NO: 70)
CᵢMFVPCᵢᵢAVREILGLCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 4 amino acids and the other of which consists of 8 amino acids, the molecular scaffold is TATB and the bicyclic peptide ligand additionally comprises N- and/or C-terminal additions and comprises an amino acid sequence which is selected from:
A-(SEQ ID NO: 3)-A (herein referred to as BCY15334);
A-(SEQ ID NO: 15)-A (herein referred to as BCY15244);
A-(SEQ ID NO: 16)-A (herein referred to as BCY15245);
A-(SEQ ID NO: 17)-A (herein referred to as BCY15246);
A-(SEQ ID NO: 18)-A (herein referred to as BCY15247);
A-(SEQ ID NO: 19)-A (herein referred to as BCY15248);
A-(SEQ ID NO: 20)-A (herein referred to as BCY15249);
A-(SEQ ID NO: 21)-A (herein referred to as BCY15250);
A-(SEQ ID NO: 26)-A (herein referred to as BCY15255);
A-(SEQ ID NO: 48)-A (herein referred to as BCY15354);
A-(SEQ ID NO: 48)-A (herein referred to as BCY16534);
A-(SEQ ID NO: 48)-AK (herein referred to as BCY16896);
A-(SEQ ID NO: 49)-A (herein referred to as BCY15355); and
A-(SEQ ID NO: 50)-A (herein referred to as BCY15356).

In a still yet further embodiment, said loop sequences comprise three reactive groups separated by two loop sequences one of which consists of 4 amino acids and the other of which consists of 8 amino acids, the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 3)-A-[Sar₆]-[KFl] (herein referred to as BCY15301);
A-(SEQ ID NO: 15)-A-[Sar₆]-[KFl] (herein referred to as BCY15307);
A-(SEQ ID NO: 17)-A-[Sar₆]-[KFl] (herein referred to as BCY15308);
A-(SEQ ID NO: 19)-A-[Sar₆]-[KFl] (herein referred to as BCY15309);
A-(SEQ ID NO: 48)-A-[Sar₆]-[KFl] (herein referred to as BCY15324);
A-(SEQ ID NO: 49)-A-[Sar₆]-[KFl] (herein referred to as BCY15325); and
A-(SEQ ID NO: 50)-A-[Sar₆]-[KFl] (herein referred to as BCY15326).

In a yet further embodiment, said loop sequences comprise three reactive groups separated by two loop sequences one of which consists of 4 amino acids and the other of which consists of 8 amino acids, the molecular scaffold is TATA, the bicyclic peptide ligand additionally comprises N- and/or C-terminal additions and comprises an amino acid sequence which is selected from:
A-(SEQ ID NO: 3)-A (herein referred to as BCY15232);
A-(SEQ ID NO: 4)-A (herein referred to as BCY15233);
A-(SEQ ID NO: 5)-A (herein referred to as BCY15234);
A-(SEQ ID NO: 14)-A (herein referred to as BCY15243);
A-(SEQ ID NO: 44)-A (herein referred to as BCY15350);
A-(SEQ ID NO: 68)-A (herein referred to as BCY15374);
A-(SEQ ID NO: 69)-A (herein referred to as BCY15375); and
A-(SEQ ID NO: 70)-A (herein referred to as BCY15376).

In a still yet further embodiment, said loop sequences comprise three reactive groups separated by two loop sequences one of which consists of 4 amino acids and the other of which consists of 8 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 3)-A-[Sar₆]-[KFl] (herein referred to as BCY15300);
A-(SEQ ID NO: 5)-A-[Sar₆]-[KFl] (herein referred to as BCY15302); and
A-(SEQ ID NO: 70)-A-[Sar₆]-[KFl] (herein referred to as BCY15330).

In a yet further embodiment, said loop sequences comprise three reactive groups separated by two loop sequences one of which consists of 4 amino acids and the other of which consists of 8 amino acids, the molecular scaffold is TBMT and the bicyclic peptide ligand additionally comprises N- and/or C-terminal additions and comprises an amino acid sequence which is:
A-(SEQ ID NO: 48)-AK (herein referred to as BCY16986).

In an alternative embodiment, said loop sequences comprise three reactive groups separated by two loop sequences one of which consists of 6 amino acids and the other of which consists of 3 amino acids.

In a further embodiment, said loop sequences comprise three reactive groups separated by two loop sequences one of which consists of 6 amino acids and the other of which consists of 3 amino acids and the bicyclic peptide ligand comprises an amino acid sequence which is selected from:

(SEQ ID NO: 71)
CᵢTLMDPWCᵢᵢLLKCᵢᵢᵢ;
(SEQ ID NO: 72)
CᵢKIHDWTCᵢᵢLLRCᵢᵢᵢ;
and
(SEQ ID NO: 79)
CᵢIPLDWTCᵢᵢMIACᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 6 amino acids and the other of which consists of 3 amino acids, the molecular scaffold is TATA and the bicyclic peptide ligand additionally comprises N- and/or C-terminal additions and comprises an amino acid sequence which is selected from:
A-(SEQ ID NO: 71)-A (herein referred to as BCY15377); and
A-(SEQ ID NO: 72)-A (herein referred to as BCY15378).

In a still yet further embodiment, said loop sequences comprise three reactive groups separated by two loop sequences one of which consists of 6 amino acids and the other of which consists of 3 amino acids, the molecular scaffold is TATA, the bicyclic peptide additionally comprises N- and/or C-terminal additions and a labelling moiety, such as fluorescein (Fl), and comprises an amino acid sequence which is:
A-(SEQ ID NO: 71)-A-[Sar₆]-[KFl] (herein referred to as BCY15331).

In an alternative embodiment, said loop sequences comprise three reactive groups separated by two loop sequences one of which consists of 6 amino acids and the other of which consists of 3 amino acids, the molecular scaffold is TATB, the bicyclic peptide ligand additionally comprises N- and/or C-terminal additions and comprises an amino acid sequence which is selected from:
Ac-(SEQ ID NO: 79) (herein referred to as BCY16991);
A-(SEQ ID NO: 79)-A (herein referred to as BCY15446);
A-(SEQ ID NO: 79)-AK (herein referred to as BCY16994); and
Ac-(SEQ ID NO: 79)-K (herein referred to as BCY18654).

In a further alternative embodiment, said loop sequences comprise three reactive groups separated by two loop sequences one of which consists of 6 amino acids and the other of which consists of 3 amino acids, the molecular scaffold is TATB, the bicyclic peptide ligand additionally comprises N- and/or C-terminal additions and comprises an amino acid sequence which is selected from:
Ac-(SEQ ID NO: 79) (herein referred to as BCY16991);
A-(SEQ ID NO: 79)-A (herein referred to as BCY15446); and
A-(SEQ ID NO: 79)-AK (herein referred to as BCY16994).

In an alternative embodiment, said loop sequences comprise three reactive groups separated by two loop sequences one of which consists of 6 amino acids and the other of which consists of 4 amino acids.

In a further embodiment, said loop sequences comprise three reactive groups separated by two loop sequences one of which consists of 6 amino acids and the other of which consists of 4 amino acids and the bicyclic peptide ligand comprises an amino acid sequence which is:

(SEQ ID NO: 11)
CᵢEYQGPHCᵢᵢYRLYCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 6 amino acids and the other of which consists of 4 amino acids, the molecular scaffold is TATA, the bicyclic peptide ligand additionally comprises N- and/or C-terminal additions and comprises an amino acid sequence which is:
A-(SEQ ID NO: 11)-A (herein referred to as BCY15240).

In a still yet further embodiment, said loop sequences comprise three reactive groups separated by two loop sequences one of which consists of 6 amino acids and the other of which consists of 4 amino acids, the molecular scaffold is TATA, the bicyclic peptide additionally comprises N- and/or C-terminal additions and a labelling moiety, such as fluorescein (Fl), and comprises an amino acid sequence which is:
A-(SEQ ID NO: 11)-A-[Sar₆]-[KFl] (herein referred to as BCY15304).

In an alternative embodiment, said loop sequences comprise three reactive groups separated by two loop sequences one of which consists of 7 amino acids and the other of which consists of 2 amino acids.

In a further embodiment, said loop sequences comprise three reactive groups separated by two loop sequences one of which consists of 7 amino acids and the other of which consists of 2 amino acids and the bicyclic peptide ligand comprises an amino acid sequence which is selected from:

(SEQ ID NO: 2)
CᵢEDHDWVYCᵢᵢSTCᵢᵢᵢ;
(SEQ ID NO: 23)
CᵢAPWNYFRCᵢᵢDLCᵢᵢᵢ;
(SEQ ID NO: 25)
CᵢLTPEDIWCᵢᵢMLCᵢᵢᵢ;
(SEQ ID NO: 28)
CᵢENPVDIWCᵢᵢVLCᵢᵢᵢ;
(SEQ ID NO: 46)
CᵢVFTTVWDCᵢᵢLACᵢᵢᵢ;
(SEQ ID NO: 51)
CᵢYDPIDVWCᵢᵢMMCᵢᵢᵢ;
(SEQ ID NO: 52)
CᵢASYDDFWCᵢᵢVLCᵢᵢᵢ;
(SEQ ID NO: 53)
CᵢDLTQHWTCᵢᵢILCᵢᵢᵢ;
(SEQ ID NO: 54)
CᵢSEISDVWCᵢᵢMLCᵢᵢᵢ;
(SEQ ID NO: 55)
CᵢPTPVDIWCᵢᵢMLCᵢᵢᵢ;
(SEQ ID NO: 73)
CᵢEQNGWIYCᵢᵢSTCᵢᵢᵢ;
(SEQ ID NO: 74)
CᵢTDRSWIFCᵢᵢSTCᵢᵢᵢ;
and
(SEQ ID NO: 75)
CᵢPNISWIYCᵢᵢSTCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 7 amino acids and the other of which consists of 2 amino acids, the molecular scaffold is TATA, the bicyclic peptide ligand additionally comprises N- and/or C-terminal additions and comprises an amino acid sequence which is selected from:
A-(SEQ ID NO: 2)-A (herein referred to as BCY15231);
Ac-(SEQ ID NO: 2) (herein referred to as BCY16987);
A-(SEQ ID NO: 2)-AK (herein referred to as BCY16990);
A-(SEQ ID NO: 46)-A (herein referred to as BCY15352);
A-(SEQ ID NO: 73)-A (herein referred to as BCY15379);
A-(SEQ ID NO: 74)-A (herein referred to as BCY15380); and
A-(SEQ ID NO: 75)-A (herein referred to as BCY15381).

In a still yet further embodiment, said loop sequences comprise three reactive groups separated by two loop sequences one of which consists of 7 amino acids and the other of which consists of 2 amino acids, the molecular scaffold is TATA, the bicyclic peptide ligand additionally comprises N- and/or C-terminal additions and comprises an amino acid sequence which is selected from:
A-(SEQ ID NO: 2)-A (herein referred to as BCY15231);
Ac-(SEQ ID NO: 2) (herein referred to as BCY16987);
A-(SEQ ID NO: 46)-A (herein referred to as BCY15352);
A-(SEQ ID NO: 73)-A (herein referred to as BCY15379);
A-(SEQ ID NO: 74)-A (herein referred to as BCY15380); and
A-(SEQ ID NO: 75)-A (herein referred to as BCY15381).

In a still yet further embodiment, said loop sequences comprise three reactive groups separated by two loop sequences one of which consists of 7 amino acids and the other of which consists of 2 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 2)-A-[Sar₆]-[KFl] (herein referred to as BCY15299); and
A-(SEQ ID NO: 74)-A-[Sar₆]-[KFl] (herein referred to as BCY15332).

In a yet further embodiment, said loop sequences comprise three reactive groups separated by two loop sequences one of which consists of 7 amino acids and the other of which consists of 2 amino acids, the molecular scaffold is TATB, the bicyclic peptide ligand additionally comprises N- and/or C-terminal additions and comprises an amino acid sequence which is selected from:
A-(SEQ ID NO: 23)-A (herein referred to as BCY15252);
A-(SEQ ID NO: 25)-A (herein referred to as BCY15254);
A-(SEQ ID NO: 28)-A (herein referred to as BCY15257);
A-(SEQ ID NO: 51)-A (herein referred to as BCY15357);
A-(SEQ ID NO: 52)-A (herein referred to as BCY15358);
A-(SEQ ID NO: 53)-A (herein referred to as BCY15359);
A-(SEQ ID NO: 54)-A (herein referred to as BCY15360); and
A-(SEQ ID NO: 55)-A (herein referred to as BCY15361).

In a still yet further embodiment, said loop sequences comprise three reactive groups separated by two loop sequences one of which consists of 7 amino acids and the other of which consists of 2 amino acids, the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 23)-A-[Sar₆]-[KFl] (herein referred to as BCY15311);
A-(SEQ ID NO: 25)-A-[Sar₆]-[KFl] (herein referred to as BCY15312); and
A-(SEQ ID NO: 53)-A-[Sar₆]-[KFl] (herein referred to as BCY15327).

In an alternative embodiment, said loop sequences comprise three reactive groups separated by two loop sequences one of which consists of 7 amino acids and the other of which consists of 3 amino acids.

In a further embodiment, said loop sequences comprise three reactive groups separated by two loop sequences one of which consists of 7 amino acids and the other of which consists of 3 amino acids and the bicyclic peptide ligand comprises an amino acid sequence which is selected from:

(SEQ ID NO: 22; herein referred to as
BCY16534 when complexed with TATB)
CᵢASPDNPVCᵢᵢRFYCᵢᵢᵢ;
(SEQ ID NO: 24; herein referred to as
BCY16540 when complexed with TATB)
CᵢYNHANPVCᵢᵢRYYCᵢᵢᵢ;
(SEQ ID NO: 27)
CᵢDLFLHELCᵢᵢDMPCᵢᵢᵢ;
(SEQ ID NO: 31)
CᵢNKQNWRYCᵢᵢYLTCᵢᵢᵢ;
(SEQ ID NO: 56)
CᵢHPWSALFCᵢᵢNYPCᵢᵢᵢ;
(SEQ ID NO: 57)
CᵢYAPDNPVCᵢᵢRMYCᵢᵢᵢ;
(SEQ ID NO: 76)
CᵢGILADPFCᵢᵢLISCᵢᵢᵢ;
(SEQ ID NO: 89)
CᵢYNHANPVCᵢᵢ[Agb]YYCᵢᵢᵢ;
(SEQ ID NO: 90)
CᵢASPDNPVCᵢᵢ[Agb]FYCᵢᵢᵢ;
(SEQ ID NO: 91)
CᵢASPDNPVCᵢᵢ[Arg(Me)]FYCᵢᵢᵢ;
(SEQ ID NO: 92)
CᵢCASPDNPVCᵢᵢ[HArg]FYCᵢᵢᵢ;
(SEQ ID NO: 93)
CᵢANPDNPVCᵢᵢRFYCᵢᵢᵢ;
(SEQ ID NO: 94)
CᵢRNPDNPVCᵢᵢRFYCᵢᵢᵢ;
(SEQ ID NO: 95)
CᵢHNPSNPVCᵢᵢRFYCᵢᵢᵢ;
(SEQ ID NO: 96)
CᵢVNKHNPVCᵢᵢRFYCᵢᵢᵢ;
(SEQ ID NO: 97)
CᵢVNAENPVCᵢᵢRFYCᵢᵢᵢ;
(SEQ ID NO: 98)
CᵢQNPGNPVCᵢᵢRFYCᵢᵢᵢ;
(SEQ ID NO: 99)
CᵢMNPDNPVCᵢᵢRFYCᵢᵢᵢ;
(SEQ ID NO: 100)
CᵢYNQENPVCᵢᵢRFYCᵢᵢᵢ;
(SEQ ID NO: 101)
CᵢNNPANPVCᵢᵢRFYCᵢᵢᵢ;
(SEQ ID NO: 102)
CᵢFNIDNPVCᵢᵢRFYCᵢᵢᵢ;
(SEQ ID NO: 103)
CᵢSNPENPVCᵢᵢRFYCᵢᵢᵢ;
(SEQ ID NO: 104)
CᵢMNEDNPVCᵢᵢRFYCᵢᵢᵢ;
(SEQ ID NO: 105)
CᵢMNEANPVCᵢᵢRFYCᵢᵢᵢ;
(SEQ ID NO: 106)
CᵢHNLDNPVCᵢᵢRFYCᵢᵢᵢ;
(SEQ ID NO: 107)
CᵢANHDNPVCᵢᵢRFYCᵢᵢᵢ;
(SEQ ID NO: 108)
CᵢKNYDNPVCᵢᵢRFYCᵢᵢᵢ;
(SEQ ID NO: 109)
CᵢENMDNPVCᵢᵢRFYCᵢᵢᵢ;
(SEQ ID NO: 110)
CᵢMNTDNPVCᵢᵢRFYCᵢᵢᵢ;
(SEQ ID NO: 111)
CᵢLNVDNPVCᵢᵢRFYCᵢᵢᵢ;
(SEQ ID NO: 112)
CᵢLNPDNPVCᵢᵢRFYCᵢᵢᵢ;
(SEQ ID NO: 113)
CᵢYNHANPVCᵢᵢ[HArg]YYCᵢᵢᵢ;
and
(SEQ ID NO: 114)
CᵢYNHANPVCᵢᵢ[Arg(Me)]YYCᵢᵢᵢ;

wherein Cᵢ, Cᵢᵢ and Cᵢᵢᵢ, represent first, second and third cysteine residues, respectively, Agb represents 2-amino-4-guanidinobutyric acid, Arg(Me) represents d-N methyl arginine, HArg represents homoarginine, or a pharmaceutically acceptable salt thereof.

In a yet further embodiment, said loop sequences comprise three reactive groups separated by two loop sequences one of which consists of 7 amino acids and the other of which consists of 3 amino acids, the molecular scaffold is TATB, the bicyclic peptide ligand additionally comprises N- and/or C-terminal additions and comprises an amino acid sequence which is selected from:
A-(SEQ ID NO: 22)-A (herein referred to as BCY15251);
Ac-A-(SEQ ID NO: 22)-A (herein referred to as BCY16538);
Ac-(SEQ ID NO: 22) (herein referred to as BCY15576);
Ac-A-(SEQ ID NO: 22)-AK (herein referred to as BCY16982);
Ac-A-(SEQ ID NO: 24)-A (herein referred to as BCY16545);
Ac-(SEQ ID NO: 24) (herein referred to as BCY16544);
A-(SEQ ID NO: 24)-A (herein referred to as BCY15522);
A-(SEQ ID NO: 27)-A (herein referred to as BCY15256);
A-(SEQ ID NO: 56)-A (herein referred to as BCY15362);
A-(SEQ ID NO: 57)-A (herein referred to as BCY15363);
A-(SEQ ID NO: 89)-A (herein referred to as BCY16541);
A-(SEQ ID NO: 90)-A (herein referred to as BCY16535);
A-(SEQ ID NO: 91)-A (herein referred to as BCY16536);
A-(SEQ ID NO: 92)-A (herein referred to as BCY16537);
Ac-(SEQ ID NO: 93) (herein referred to as BCY16903);
Ac-(SEQ ID NO: 93)-K (herein referred to as BCY18579); Ac-(SEQ ID NO: 94) (herein referred to as BCY16905);
Ac-(SEQ ID NO: 95) (herein referred to as BCY16906);
Ac-(SEQ ID NO: 96) (herein referred to as BCY16911);
Ac-(SEQ ID NO: 97) (herein referred to as BCY16913);
Ac-(SEQ ID NO: 98) (herein referred to as BCY16915);
Ac-(SEQ ID NO: 99) (herein referred to as BCY16917);
Ac-(SEQ ID NO: 100) (herein referred to as BCY16918);
Ac-(SEQ ID NO: 101) (herein referred to as BCY16921);
Ac-(SEQ ID NO: 102) (herein referred to as BCY16912);
Ac-(SEQ ID NO: 103) (herein referred to as BCY16914);
Ac-(SEQ ID NO: 104) (herein referred to as BCY16916);
Ac-(SEQ ID NO: 105) (herein referred to as BCY16919);
Ac-(SEQ ID NO: 106) (herein referred to as BCY16920);
Ac-(SEQ ID NO: 107) (herein referred to as BCY16902);
Ac-(SEQ ID NO: 108) (herein referred to as BCY16904);
Ac-(SEQ ID NO: 109) (herein referred to as BCY16907);
Ac-(SEQ ID NO: 110) (herein referred to as BCY16908);
Ac-(SEQ ID NO: 111) (herein referred to as BCY16909);
Ac-(SEQ ID NO: 112) (herein referred to as BCY16910);
A-(SEQ ID NO: 113)-A (herein referred to as BCY16543); and
A-(SEQ ID NO: 114)-A (herein referred to as BCY16542).

In a yet further embodiment, said loop sequences comprise three reactive groups separated by two loop sequences one of which consists of 7 amino acids and the other of which consists of 3 amino acids, the molecular scaffold is TATB, the bicyclic peptide ligand additionally comprises N- and/or C-terminal additions and comprises an amino acid sequence which is selected from:
A-(SEQ ID NO: 22)-A (herein referred to as BCY15251);
Ac-A-(SEQ ID NO: 22)-A (herein referred to as BCY16538);
Ac-(SEQ ID NO: 22) (herein referred to as BCY15576);
Ac-A-(SEQ ID NO: 24)-A (herein referred to as BCY16545);
Ac-(SEQ ID NO: 24) (herein referred to as BCY16544);
A-(SEQ ID NO: 24)-A (herein referred to as BCY15522);
A-(SEQ ID NO: 27)-A (herein referred to as BCY15256);
A-(SEQ ID NO: 56)-A (herein referred to as BCY15362);
A-(SEQ ID NO: 57)-A (herein referred to as BCY15363);
A-(SEQ ID NO: 89)-A (herein referred to as BCY16541);
A-(SEQ ID NO: 90)-A (herein referred to as BCY16535);
A-(SEQ ID NO: 91)-A (herein referred to as BCY16536);
A-(SEQ ID NO: 92)-A (herein referred to as BCY16537);
Ac-(SEQ ID NO: 93) (herein referred to as BCY16903);
Ac-(SEQ ID NO: 94) (herein referred to as BCY16905);
Ac-(SEQ ID NO: 95) (herein referred to as BCY16906);
Ac-(SEQ ID NO: 96) (herein referred to as BCY16911);
Ac-(SEQ ID NO: 97) (herein referred to as BCY16913);
Ac-(SEQ ID NO: 98) (herein referred to as BCY16915);
Ac-(SEQ ID NO: 99) (herein referred to as BCY16917);
Ac-(SEQ ID NO: 100) (herein referred to as BCY16918);
Ac-(SEQ ID NO: 101) (herein referred to as BCY16921);
Ac-(SEQ ID NO: 102) (herein referred to as BCY16912);
Ac-(SEQ ID NO: 103) (herein referred to as BCY16914);
Ac-(SEQ ID NO: 104) (herein referred to as BCY16916);
Ac-(SEQ ID NO: 105) (herein referred to as BCY16919);
Ac-(SEQ ID NO: 106) (herein referred to as BCY16920);
Ac-(SEQ ID NO: 107) (herein referred to as BCY16902);
Ac-(SEQ ID NO: 108) (herein referred to as BCY16904);
Ac-(SEQ ID NO: 109) (herein referred to as BCY16907);
Ac-(SEQ ID NO: 110) (herein referred to as BCY16908);
Ac-(SEQ ID NO: 111) (herein referred to as BCY16909);
Ac-(SEQ ID NO: 112) (herein referred to as BCY16910);
A-(SEQ ID NO: 113)-A (herein referred to as BCY16543); and
A-(SEQ ID NO: 114)-A (herein referred to as BCY16542).

In a still yet further embodiment, said loop sequences comprise three reactive groups separated by two loop sequences one of which consists of 7 amino acids and the other of which consists of 3 amino acids, the molecular scaffold is TATB, the bicyclic peptide additionally comprises N- and/or C-terminal additions and a labelling moiety, such as fluorescein (Fl), and comprises an amino acid sequence which is selected from:
A-(SEQ ID NO: 22)-A-[Sar₆]-[KFl] (herein referred to as BCY15310);
A-(SEQ ID NO: 27)-A-[Sar₆]-[KFl] (herein referred to as BCY15313); and
A-(SEQ ID NO: 56)-A-[Sar₆]-[KFl] (herein referred to as BCY15328).

In a yet further embodiment, said loop sequences comprise three reactive groups separated by two loop sequences one of which consists of 7 amino acids and the other of which consists of 3 amino acids, the molecular scaffold is TBMT, the bicyclic peptide ligand additionally comprises N- and/or C-terminal additions and comprises an amino acid sequence which is:
A-(SEQ ID NO: 31)-A (herein referred to as BCY15315).

In a still yet further embodiment, said loop sequences comprise three reactive groups separated by two loop sequences one of which consists of 7 amino acids and the other of which consists of 3 amino acids, the molecular scaffold is TBMT, the bicyclic peptide additionally comprises N- and/or C-terminal additions and a labelling moiety, such as fluorescein (Fl), and comprises an amino acid sequence which is:
A-(SEQ ID NO: 31)-A-[Sar₆]-[KFl] (herein referred to as BCY15313).

In a yet further embodiment, said loop sequences comprise three reactive groups separated by two loop sequences one of which consists of 7 amino acids and the other of which consists of 3 amino acids, the molecular scaffold is TATA, the bicyclic peptide ligand additionally comprises N- and/or C-terminal additions and comprises an amino acid sequence which is:
A-(SEQ ID NO: 76)-A (herein referred to as BCY15382).

In a still yet further embodiment, said loop sequences comprise three reactive groups separated by two loop sequences one of which consists of 7 amino acids and the other of which consists of 3 amino acids, the molecular scaffold is TATA, the bicyclic peptide additionally comprises N- and/or C-terminal additions and a labelling moiety, such as fluorescein (Fl), and comprises an amino acid sequence which is:
A-(SEQ ID NO: 76)-A-[Sar₆]-[KFl] (herein referred to as BCY15333).

In an alternative embodiment, said loop sequences comprise three reactive groups separated by two loop sequences one of which consists of 7 amino acids and the other of which consists of 5 amino acids.

In a further embodiment, said loop sequences comprise three reactive groups separated by two loop sequences one of which consists of 7 amino acids and the other of which consists of 5 amino acids and the bicyclic peptide ligand comprises an amino acid sequence which is selected from:

(SEQ ID NO: 32)
CᵢTTSEKVKCᵢᵢLQRHPCᵢᵢᵢ;
(SEQ ID NO: 33)
CᵢQPDMRIKCᵢᵢLQRVACᵢᵢᵢ;
(SEQ ID NO: 34)
CᵢSSNNRIKCᵢᵢLQRVTCᵢᵢᵢ;
and
(SEQ ID NO: 35)
CᵢKEKTTIGCᵢᵢLMAGICᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 7 amino acids and the other of which consists of 5 amino acids, the molecular scaffold is TBMT, the bicyclic peptide ligand additionally comprises N- and/or C-terminal additions and comprises an amino acid sequence which is selected from:
A-(SEQ ID NO: 32)-A (herein referred to as BCY15338);
A-(SEQ ID NO: 33)-A (herein referred to as BCY15339);
A-(SEQ ID NO: 34)-A (herein referred to as BCY15340); and
A-(SEQ ID NO: 35)-A (herein referred to as BCY15341).

In a still yet further embodiment, said loop sequences comprise three reactive groups separated by two loop sequences one of which consists of 7 amino acids and the other of which consists of 5 amino acids, the molecular scaffold is TBMT, the bicyclic peptide additionally comprises N- and/or C-terminal additions and a labelling moiety, such as fluorescein (Fl), and comprises an amino acid sequence which is selected from:
A-(SEQ ID NO: 32)-A-[Sar₆]-[KFl] (herein referred to as BCY15316); and
A-(SEQ ID NO: 33)-A-[Sar₆]-[KFl] (herein referred to as BCY15317).

In an alternative embodiment, said loop sequences comprise three reactive groups separated by two loop sequences one of which consists of 8 amino acids and the other of which consists of 2 amino acids.

In a further embodiment, said loop sequences comprise three reactive groups separated by two loop sequences one of which consists of 8 amino acids and the other of which consists of 2 amino acids and the bicyclic peptide ligand comprises an amino acid sequence which is selected from:

(SEQ ID NO: 12)
CᵢGRDSSWIYCᵢᵢSTCᵢᵢᵢ;
(SEQ ID NO: 13)
CᵢRGTPAWKACᵢᵢAICᵢᵢᵢ;
(SEQ ID NO: 36)
CᵢPFPSGFGTCᵢᵢTFCᵢᵢᵢ;
(SEQ ID NO: 37; herein referred to as
BCY16312 when complexed with TBMT)
CᵢPYVAGRGTCᵢᵢLLCᵢᵢᵢ;
(SEQ ID NO: 38)
CᵢPYPRGTGSCᵢᵢTFCᵢᵢᵢ;
(SEQ ID NO: 39)
CᵢLYPPGKGTCᵢᵢLLCᵢᵢᵢ;
(SEQ ID NO: 40)
CᵢPSPAGRGTCᵢᵢLLCᵢᵢᵢ;
(SEQ ID NO: 41)
CᵢPATIGRGPCᵢᵢTFCᵢᵢᵢ;
(SEQ ID NO: 77)
CᵢPEANSWVYCᵢᵢSTCᵢᵢᵢ;
(SEQ ID NO: 78)
CᵢAPTSGWIYCᵢᵢSTCᵢᵢᵢ;
(SEQ ID NO: 80)
CᵢPYVAG[Agb]GTCᵢᵢLLCᵢᵢᵢ;
(SEQ ID NO: 81)
CᵢPYVAG[Arg(Me)]GTCᵢᵢLLCᵢᵢᵢ;
(SEQ ID NO: 82)
CᵢPYVAGRGTCᵢᵢL[Cba]Cᵢᵢᵢ;
(SEQ ID NO: 83)
CᵢPYVAGRGTCᵢᵢ[Cba]LCᵢᵢᵢ;
(SEQ ID NO: 84)
CᵢPYVAGR[dA]TCᵢᵢLLCᵢᵢᵢ;
(SEQ ID NO: 85)
CᵢPYVAG[HArg]GTCᵢᵢLLCᵢᵢᵢ;
(SEQ ID NO: 86)
CᵢPYVAGRGTCᵢᵢL[tBuAla]Cᵢᵢᵢ;
(SEQ ID NO: 87)
CᵢPYVAGRGTCᵢᵢ[tBuAla]LCᵢᵢᵢ;
(SEQ ID NO: 88)
CᵢPYVAG[Agb][dA]TCᵢᵢL[tBuAla]Cᵢᵢᵢ;
and
(SEQ ID NO: 115)
CᵢPYVAG[HArg][dA]TCᵢᵢL[tBuAla]Cᵢᵢᵢ;

wherein Cᵢ, Cᵢᵢ and Cᵢᵢᵢ represent first, second and third cysteine residues, respectively, Agb represents 2-amino-4-guanidinobutyric acid, Arg(Me) represents d-N methyl arginine, Cba represents β-cyclobutylalanine, HArg represents homoarginine, tBuAla represents t-butyl-alanine, or a pharmaceutically acceptable salt thereof.

In a yet further embodiment, said loop sequences comprise three reactive groups separated by two loop sequences one of which consists of 8 amino acids and the other of which consists of 2 amino acids and the bicyclic peptide ligand comprises an amino acid sequence which is selected from:

(SEQ ID NO: 12)
CᵢGRDSSWIYCᵢᵢSTCᵢᵢᵢ;
(SEQ ID NO: 13)
CᵢRGTPAWKACᵢᵢAICᵢᵢᵢ;
(SEQ ID NO: 36)
CᵢPFPSGFGTCᵢᵢTFCᵢᵢᵢ;
(SEQ ID NO: 37; herein referred to as
BCY16312 when complexed with TBMT)
CᵢPYVAGRGTCᵢᵢLLCᵢᵢᵢ;
(SEQ ID NO: 38)
CᵢPYPRGTGSCᵢᵢTFCᵢᵢᵢ;
(SEQ ID NO: 39)
CᵢLYPPGKGTCᵢᵢLLCᵢᵢᵢ;
(SEQ ID NO: 40)
CᵢPSPAGRGTCᵢᵢLLCᵢᵢᵢ;
(SEQ ID NO: 41)
CᵢPATIGRGPCᵢᵢTFCᵢᵢᵢ;
(SEQ ID NO: 77)
CᵢPEANSWVYCᵢᵢSTCᵢᵢᵢ;
(SEQ ID NO: 78)
CᵢAPTSGWIYCᵢᵢSTCᵢᵢᵢ;
(SEQ ID NO: 80)
CᵢPYVAG[Agb]GTCᵢᵢLLCᵢᵢᵢ;
(SEQ ID NO: 81)
CᵢPYVAG[Arg(Me)]GTCᵢᵢLLCᵢᵢᵢ;
(SEQ ID NO: 82)
CᵢPYVAGRGTCᵢᵢL[Cba]Cᵢᵢᵢ;
(SEQ ID NO: 83)
CᵢPYVAGRGTCᵢᵢ[Cba]LCᵢᵢᵢ;
(SEQ ID NO: 84)
CᵢPYVAGR[dA]TCᵢᵢLLCᵢᵢᵢ;
(SEQ ID NO: 85)
CᵢPYVAG[HArg]GTCᵢᵢLLCᵢᵢᵢ;
(SEQ ID NO: 86)
CᵢPYVAGRGTCᵢᵢL[tBuAla]Cᵢᵢᵢ;
(SEQ ID NO: 87)
CᵢPYVAGRGTCᵢᵢ[tBuAla]LCᵢᵢᵢ;
and
(SEQ ID NO: 88)
CᵢPYVAG[Agb][dA]TCᵢᵢL[tBuAla]Cᵢᵢᵢ;

wherein Cᵢ, Cᵢᵢ and Cᵢᵢᵢ represent first, second and third cysteine residues, respectively, Agb represents 2-amino-4-guanidinobutyric acid, Arg(Me) represents d-N methyl arginine, Cba represents β-cyclobutylalanine, HArg represents homoarginine, tBuAla represents t-butyl-alanine, or a pharmaceutically acceptable salt thereof.

In a yet further embodiment, said loop sequences comprise three reactive groups separated by two loop sequences one of which consists of 8 amino acids and the other of which consists of 2 amino acids, the molecular scaffold is TATA, the bicyclic peptide ligand additionally comprises N- and/or C-terminal additions and comprises an amino acid sequence which is selected from:
A-(SEQ ID NO: 12)-A (herein referred to as BCY15241);
A-(SEQ ID NO: 13)-A (herein referred to as BCY15242);
A-(SEQ ID NO: 77)-A (herein referred to as BCY15383); and
A-(SEQ ID NO: 78)-A (herein referred to as BCY15384).

In a still yet further embodiment, said loop sequences comprise three reactive groups separated by two loop sequences one of which consists of 8 amino acids and the other of which consists of 2 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 12)-A-[Sar₆]-[KFl] (herein referred to as BCY15305); and
A-(SEQ ID NO: 13)-A-[Sar₆]-[KFl] (herein referred to as BCY15306).

In a yet further embodiment, said loop sequences comprise three reactive groups separated by two loop sequences one of which consists of 8 amino acids and the other of which consists of 2 amino acids, the molecular scaffold is TBMT, the bicyclic peptide ligand additionally comprises N- and/or C-terminal additions and comprises an amino acid sequence which is selected from:
A-(SEQ ID NO: 36)-A (herein referred to as BCY15342);
A-(SEQ ID NO: 37)-A (herein referred to as BCY15343);
Ac-A-(SEQ ID NO: 37)-A (herein referred to as BCY16322);
Ac-(SEQ ID NO: 37) (herein referred to as BCY16323);
A-(SEQ ID NO: 38)-A (herein referred to as BCY15344);
A-(SEQ ID NO: 39)-A (herein referred to as BCY15345);
A-(SEQ ID NO: 40)-A (herein referred to as BCY15346);
A-(SEQ ID NO: 41)-A (herein referred to as BCY15347);
A-(SEQ ID NO: 80)-A (herein referred to as BCY16313);
A-(SEQ ID NO: 81)-A (herein referred to as BCY16314);
A-(SEQ ID NO: 82)-A (herein referred to as BCY16315);
A-(SEQ ID NO: 83)-A (herein referred to as BCY16316);
A-(SEQ ID NO: 84)-A (herein referred to as BCY16318);
A-(SEQ ID NO: 85)-A (herein referred to as BCY16319);
A-(SEQ ID NO: 86)-A (herein referred to as BCY16320);
A-(SEQ ID NO: 87)-A (herein referred to as BCY16321);
Ac-(SEQ ID NO: 88) (herein referred to as BCY16591);
Ac-(SEQ ID NO: 88)-[K(PYA)] (herein referred to as BCY16592);
Ac-(SEQ ID NO: 88)-K (herein referred to as BCY17018); and
Ac-(SEQ ID NO: 115) (herein referred to as BCY18024),
   wherein PYA represents pentynoic acid.

In a still yet further embodiment, said loop sequences comprise three reactive groups separated by two loop sequences one of which consists of 8 amino acids and the other of which consists of 2 amino acids, the molecular scaffold is TBMT, the bicyclic peptide ligand additionally comprises N- and/or C-terminal additions and comprises an amino acid sequence which is selected from:
A-(SEQ ID NO: 36)-A (herein referred to as BCY15342);
A-(SEQ ID NO: 37)-A (herein referred to as BCY15343);
Ac-A-(SEQ ID NO: 37)-A (herein referred to as BCY16322);
Ac-(SEQ ID NO: 37) (herein referred to as BCY16323);
A-(SEQ ID NO: 38)-A (herein referred to as BCY15344);
A-(SEQ ID NO: 39)-A (herein referred to as BCY15345);
A-(SEQ ID NO: 40)-A (herein referred to as BCY15346);
A-(SEQ ID NO: 41)-A (herein referred to as BCY15347);
A-(SEQ ID NO: 80)-A (herein referred to as BCY16313);
A-(SEQ ID NO: 81)-A (herein referred to as BCY16314);
A-(SEQ ID NO: 82)-A (herein referred to as BCY16315);
A-(SEQ ID NO: 83)-A (herein referred to as BCY16316);
A-(SEQ ID NO: 84)-A (herein referred to as BCY16318);
A-(SEQ ID NO: 85)-A (herein referred to as BCY16319);
A-(SEQ ID NO: 86)-A (herein referred to as BCY16320);
A-(SEQ ID NO: 87)-A (herein referred to as BCY16321); and
Ac-(SEQ ID NO: 88) (herein referred to as BCY16591).

In a still yet further embodiment, said loop sequences comprise three reactive groups separated by two loop sequences one of which consists of 8 amino acids and the other of which consists of 2 amino acids, the molecular scaffold is TBMT, the bicyclic peptide additionally comprises N- and/or C-terminal additions and a labelling moiety, such as fluorescein (Fl), and comprises an amino acid sequence which is selected from:
A-(SEQ ID NO: 37)-A-[Sar₆]-[KFl] (herein referred to as BCY15318); and
A-(SEQ ID NO: 38)-A-[Sar₆]-[KFl] (herein referred to as BCY15319).

In an alternative embodiment, said loop sequences comprise three reactive groups separated by two loop sequences one of which consists of 8 amino acids and the other of which consists of 3 amino acids.

In a further embodiment, said loop sequences comprise three reactive groups separated by two loop sequences one of which consists of 8 amino acids and the other of which consists of 3 amino acids and the bicyclic peptide ligand comprises an amino acid sequence which is selected from:

(SEQ ID NO: 45)
CᵢSNTWHWTDCᵢᵢLAECᵢᵢᵢ;
and
(SEQ ID NO: 47)
CᵢNLWNGDPWCᵢᵢLLRCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 8 amino acids and the other of which consists of 3 amino acids, the molecular scaffold is TATA, the bicyclic peptide ligand additionally comprises N- and/or C-terminal additions and comprises an amino acid sequence which is selected from:
A-(SEQ ID NO: 45)-A (herein referred to as BCY15351); and
A-(SEQ ID NO: 47)-A (herein referred to as BCY15353).

In a still yet further embodiment, said loop sequences comprise three reactive groups separated by two loop sequences one of which consists of 8 amino acids and the other of which consists of 3 amino acids, the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 45)-A-[Sar₆]-[KFl] (herein referred to as BCY15322); and
A-(SEQ ID NO: 47)-A-[Sar₆]-[KFl] (herein referred to as BCY15323).

In an alternative embodiment, said loop sequences comprise three reactive groups separated by two loop sequences one of which consists of 8 amino acids and the other of which consists of 4 amino acids.

In a further embodiment, said loop sequences comprise three reactive groups separated by two loop sequences one of which consists of 8 amino acids and the other of which consists of 4 amino acids and the bicyclic peptide ligand comprises an amino acid sequence which is selected from:

(SEQ ID NO: 42)
CᵢHQLMDIWDCᵢᵢLRPDCᵢᵢᵢ;
and
(SEQ ID NO: 43)
CᵢLTAREKIQCᵢᵢLQRRCᵢᵢᵢ;

wherein Cᵢ, Cᵢᵢ and Cᵢᵢᵢ represent first, second and third cysteine residues, respectively, or a pharmaceutically acceptable salt thereof.

In a yet further embodiment, said loop sequences comprise three reactive groups separated by two loop sequences one of which consists of 8 amino acids and the other of which consists of 4 amino acids, the molecular scaffold is TBMT, the bicyclic peptide ligand additionally comprises N- and/or C-terminal additions and comprises an amino acid sequence which is selected from:
A-(SEQ ID NO: 42)-A (herein referred to as BCY15348); and
A-(SEQ ID NO: 43)-A (herein referred to as BCY15349).

In a still yet further embodiment, said loop sequences comprise three reactive groups separated by two loop sequences one of which consists of 8 amino acids and the other of which consists of 2 amino acids, the molecular scaffold is TBMT, the bicyclic peptide additionally comprises N- and/or C-terminal additions and a labelling moiety, such as fluorescein (Fl), and comprises an amino acid sequence which is selected from:
A-(SEQ ID NO: 42)-A-[Sar₆]-[KFl] (herein referred to as BCY15320); and
A-(SEQ ID NO: 43)-A-[Sar₆]-[KFl] (herein referred to as BCY15321).

Unless defined otherwise, all technical and scientific terms used herein have the same meaning as commonly understood by those of ordinary skill in the art, such as in the arts of peptide chemistry, cell culture and phage display, nucleic acid chemistry and biochemistry. Standard techniques are used for molecular biology, genetic and biochemical methods (see Sambrook et al., Molecular Cloning: A Laboratory Manual, 3rd ed., 2001, Cold Spring Harbor Laboratory Press, Cold Spring Harbor, NY; Ausubel et al., Short Protocols in Molecular Biology (1999) 4th ed., John Wiley & Sons, Inc.), which are incorporated herein by reference.

### Nomenclature

### Numbering

When referring to amino acid residue positions within peptides of the invention, cysteine residues (Cᵢ, Cᵢᵢ and Cᵢᵢᵢ) are omitted from the numbering as they are invariant, therefore, the numbering of amino acid residues within peptides of the invention is referred to as below:

(SEQ ID NO: 1)
Cᵢ-H₁-H₂-A₃-Cᵢᵢ-P₄-I₅-L₆-T₇-G₈-W₉-Cᵢᵢᵢ.

For the purpose of this description, all bicyclic peptides are assumed to be cyclised with TATA, TATB or TBMT and yielding a tri-substituted structure. Cyclisation with TATA, TATB or TBMT occurs on the first, second and third reactive groups (i.e. Cᵢ, Cᵢᵢ, Cᵢᵢᵢ).

### Molecular Format

N- or C-terminal extensions to the bicycle core sequence are added to the left or right side of the sequence, separated by a hyphen. For example, an N-terminal βAla-Sar10-Ala tail would be denoted as:

(SEQ ID NO: X)
βAla-Sar10-A-.

### Inversed Peptide Sequences

In light of the disclosure in Nair et al (2003) J Immunol 170(3), 1362-1373, it is envisaged that the peptide sequences disclosed herein would also find utility in their retro-inverso form. For example, the sequence is reversed (i.e. N-terminus becomes C-terminus and vice versa) and their stereochemistry is likewise also reversed (i.e. D-amino acids become L-amino acids and vice versa).

### Peptide Ligands

A peptide ligand, as referred to herein, refers to a peptide covalently bound to a molecular scaffold. Typically, such peptides comprise two or more reactive groups (i.e. cysteine residues) which are capable of forming covalent bonds to the scaffold, and a sequence subtended between said reactive groups which is referred to as the loop sequence, since it forms a loop when the peptide is bound to the scaffold. In the present case, the peptides comprise at least three cysteine residues (referred to herein as Cᵢ, Cᵢᵢ and Cᵢᵢᵢ), and form at least two loops on the scaffold.

### Multimeric Binding Complexes

### Dimers

In one embodiment, the multimeric binding complex comprises a dimeric binding complex described in the following Table A:

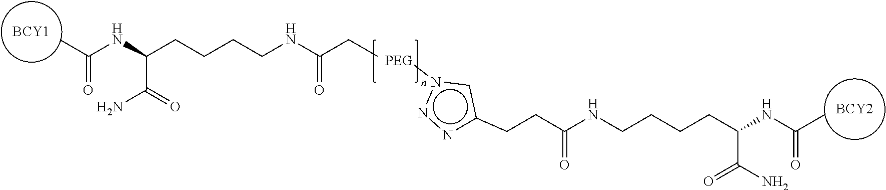

TABLE A
Exemplified Dimeric Binding Complexes of the Invention

| Multimer | | | |
|---|---|---|---|
| Compound Number | BCY1 | BCY2 | n |
| BCY17181 | BCY15318 | BCY16591 | 24 |
| BCY17182 | BCY15318 | BCY15231 | 24 |
| BCY17183 | BCY15318 | BCY15446 | 24 |
| BCY17184 | BCY15318 | BCY16994 | 24 |
| BCY17187 | BCY16986 | BCY15446 | 36 |
| BCY17195 | BCY16591 | BCY15354 | 24 |
| BCY17197 | BCY16591 | BCY15231 | 24 |
| BCY17198 | BCY16591 | BCY15446 | 24 |
| BCY17199 | BCY16591 | BCY16994 | 24 |
| BCY17188 | BCY15231 | BCY15446 | 24 |
| BCY17190 | BCY16994 | BCY15354 | 24 |
| BCY17191 | BCY17018 | BCY16982 | 24 |
| BCY17192 | BCY16990 | BCY16982 | 24 |
| BCY17196 | BCY17018 | BCY16591 | 24 |
| BCY17291 | BCY16994 | BCY16591 | 24 |
| BCY17292 | BCY16994 | BCY15231 | 24 |
| BCY17193 | BCY16994 | BCY15446 | 24 |
| BCY18653 | BCY15446 | BCY16896 | 36 |
| BCY18786 | BCY18579 | BCY18024 | 24 |
| BCY18656 | BCY18654 | BCY16591 | 24 |

### Advantages of the Peptide Ligands

Certain bicyclic peptides of the present invention have a number of advantageous properties which enable them to be considered as suitable drug-like molecules for injection, inhalation, nasal, ocular, oral or topical administration. Such advantageous properties include:
Species cross-reactivity. Certain ligands demonstrate cross-reactivity across Lipid II from different bacterial species and hence are able to treat infections caused by multiple species of bacteria. Other ligands may be highly specific for the Lipid II of certain bacterial species which may be advantageous for treating an infection without collateral damage to the beneficial flora of the patient;
Protease stability. Bicyclic peptide ligands should ideally demonstrate stability to plasma proteases, epithelial (“membrane-anchored”) proteases, gastric and intestinal proteases, lung surface proteases, intracellular proteases and the like. Protease stability should be maintained between different species such that a bicycle lead candidate can be developed in animal models as well as administered with confidence to humans;
Desirable solubility profile. This is a function of the proportion of charged and hydrophilic versus hydrophobic residues and intra/inter-molecular H-bonding, which is important for formulation and absorption purposes;
An optimal plasma half-life in the circulation. Depending upon the clinical indication and treatment regimen, it may be required to develop a bicyclic peptide for short exposure in an acute illness management setting, or develop a bicyclic peptide with enhanced retention in the circulation, and is therefore optimal for the management of more chronic disease states. Other factors driving the desirable plasma half-life are requirements of sustained exposure for maximal therapeutic efficiency versus the accompanying toxicology due to sustained exposure of the agent; and
Selectivity.

### Pharmaceutically Acceptable Salts

It will be appreciated that salt forms are within the scope of this invention, and references to peptide ligands include the salt forms of said ligands.

The salts of the present invention can be synthesized from the parent compound that contains a basic or acidic moiety by conventional chemical methods such as methods described in Pharmaceutical Salts: Properties, Selection, and Use, P. Heinrich Stahl (Editor), Camille G. Wermuth (Editor), ISBN: 3-90639-026-8, Hardcover, 388 pages, August 2002. Generally, such salts can be prepared by reacting the free acid or base forms of these compounds with the appropriate base or acid in water or in an organic solvent, or in a mixture of the two.

Acid addition salts (mono- or di-salts) may be formed with a wide variety of acids, both inorganic and organic. Examples of acid addition salts include mono- or di-salts formed with an acid selected from the group consisting of acetic, 2,2-dichloroacetic, adipic, alginic, ascorbic (e.g. L-ascorbic), L-aspartic, benzenesulfonic, benzoic, 4-acetamidobenzoic, butanoic, (+) camphoric, camphor-sulfonic, (+)-(1S)-camphor-10-sulfonic, capric, caproic, caprylic, cinnamic, citric, cyclamic, dodecylsulfuric, ethane-1,2-disulfonic, ethanesulfonic, 2-hydroxyethanesulfonic, formic, fumaric, galactaric, gentisic, glucoheptonic, D-gluconic, glucuronic (e.g. D-glucuronic), glutamic (e.g. L-glutamic), α-oxoglutaric, glycolic, hippuric, hydrohalic acids (e.g. hydrobromic, hydrochloric, hydriodic), isethionic, lactic (e.g. (+)-L-lactic, (±)-DL-lactic), lactobionic, maleic, malic, (−)-L-malic, malonic, (±)-DL-mandelic, methanesulfonic, naphthalene-2-sulfonic, naphthalene-1,5-disulfonic, 1-hydroxy-2-naphthoic, nicotinic, nitric, oleic, orotic, oxalic, palmitic, pamoic, phosphoric, propionic, pyruvic, L-pyroglutamic, salicylic, 4-amino-salicylic, sebacic, stearic, succinic, sulfuric, tannic, (+)-L-tartaric, thiocyanic, p-toluenesulfonic, undecylenic and valeric acids, as well as acylated amino acids and cation exchange resins.

One particular group of salts consists of salts formed from acetic, hydrochloric, hydriodic, phosphoric, nitric, sulfuric, citric, lactic, succinic, maleic, malic, isethionic, fumaric, benzenesulfonic, toluenesulfonic, sulfuric, methanesulfonic (mesylate), ethanesulfonic, naphthalenesulfonic, valeric, propanoic, butanoic, malonic, glucuronic and lactobionic acids. One particular salt is the hydrochloride salt. Another particular salt is the acetate salt.

If the compound is anionic, or has a functional group which may be anionic (e.g., —COOH may be —COO⁻), then a salt may be formed with an organic or inorganic base, generating a suitable cation. Examples of suitable inorganic cations include, but are not limited to, alkali metal ions such as Li⁺, Na⁺ and K⁺, alkaline earth metal cations such as Ca²⁺ and Mg²⁺, and other cations such as Al³⁺ or Zn⁺. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: methylamine, ethylamine, diethylamine, propylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

Where the peptides of the invention contain an amine function, these may form quaternary ammonium salts, for example by reaction with an alkylating agent according to methods well known to the skilled person. Such quaternary ammonium compounds are within the scope of the peptides of the invention.

### Modified Derivatives

It will be appreciated that modified derivatives of the peptide ligands as defined herein are within the scope of the present invention. Examples of such suitable modified derivatives include one or more modifications selected from: N-terminal and/or C-terminal modifications; replacement of one or more amino acid residues with one or more non-natural amino acid residues (such as replacement of one or more polar amino acid residues with one or more isosteric or isoelectronic amino acids; replacement of one or more non-polar amino acid residues with other non-natural isosteric or isoelectronic amino acids); addition of a spacer group; replacement of one or more oxidation sensitive amino acid residues with one or more oxidation resistant amino acid residues; replacement of one or more amino acid residues with an alanine, replacement of one or more L-amino acid residues with one or more D-amino acid residues; N-alkylation of one or more amide bonds within the bicyclic peptide ligand; replacement of one or more peptide bonds with a surrogate bond; peptide backbone length modification; substitution of the hydrogen on the alpha-carbon of one or more amino acid residues with another chemical group, modification of amino acids such as cysteine, lysine, glutamate/aspartate and tyrosine with suitable amine, thiol, carboxylic acid and phenol-reactive reagents so as to functionalise said amino acids, and introduction or replacement of amino acids that introduce orthogonal reactivities that are suitable for functionalisation, for example azide or alkyne-group bearing amino acids that allow functionalisation with alkyne or azide-bearing moieties, respectively.

In one embodiment, the modified derivative comprises an N-terminal and/or C-terminal modification. In a further embodiment, wherein the modified derivative comprises an N-terminal modification using suitable amino-reactive chemistry, and/or C-terminal modification using suitable carboxy-reactive chemistry. In a further embodiment, said N-terminal or C-terminal modification comprises addition of an effector group, including but not limited to a cytotoxic agent, a radiochelator or a chromophore.

In a further embodiment, the modified derivative comprises an N-terminal modification. In a further embodiment, the N-terminal modification comprises an N-terminal acetyl group. In this embodiment, the N-terminal cysteine group (the group referred to herein as Cᵢ) is capped with acetic anhydride or other appropriate reagents during peptide synthesis leading to a molecule which is N-terminally acetylated. This embodiment provides the advantage of removing a potential recognition point for aminopeptidases and avoids the potential for degradation of the bicyclic peptide.

In an alternative embodiment, the N-terminal modification comprises the addition of a molecular spacer group which facilitates the conjugation of effector groups and retention of potency of the bicyclic peptide to its target.

In a further embodiment, the modified derivative comprises a C-terminal modification. In a further embodiment, the C-terminal modification comprises an amide group. In this embodiment, the C-terminal cysteine group (the group referred to herein as Cᵢᵢᵢ) is synthesized as an amide during peptide synthesis leading to a molecule which is C-terminally amidated. This embodiment provides the advantage of removing a potential recognition point for carboxypeptidase and reduces the potential for proteolytic degradation of the bicyclic peptide.

In one embodiment, the modified derivative comprises replacement of one or more amino acid residues with one or more non-natural amino acid residues. In this embodiment, non-natural amino acids may be selected having isosteric/isoelectronic side chains which are neither recognised by degradative proteases nor have any adverse effect upon target potency.

Alternatively, non-natural amino acids may be used having constrained amino acid side chains, such that proteolytic hydrolysis of the nearby peptide bond is conformationally and sterically impeded. In particular, these concern proline analogues, bulky sidechains, C□-disubstituted derivatives (for example, aminoisobutyric acid, Aib), and cyclo amino acids, a simple derivative being amino-cyclopropylcarboxylic acid.

In one embodiment, the modified derivative comprises the addition of a spacer group. In a further embodiment, the modified derivative comprises the addition of a spacer group to the N-terminal cysteine (Cᵢ) and/or the C-terminal cysteine (Cᵢᵢᵢ).

In one embodiment, the modified derivative comprises replacement of one or more oxidation sensitive amino acid residues with one or more oxidation resistant amino acid residues.

In one embodiment, the modified derivative comprises replacement of one or more charged amino acid residues with one or more hydrophobic amino acid residues. In an alternative embodiment, the modified derivative comprises replacement of one or more hydrophobic amino acid residues with one or more charged amino acid residues. The correct balance of charged versus hydrophobic amino acid residues is an important characteristic of the bicyclic peptide ligands. For example, hydrophobic amino acid residues influence the degree of plasma protein binding and thus the concentration of the free available fraction in plasma, while charged amino acid residues (in particular arginine) may influence the interaction of the peptide with the phospholipid membranes on cell surfaces. The two in combination may influence half-life, volume of distribution and exposure of the peptide drug, and can be tailored according to the clinical endpoint. In addition, the correct combination and number of charged versus hydrophobic amino acid residues may reduce irritation at the injection site (if the peptide drug has been administered subcutaneously).

In one embodiment, the modified derivative comprises replacement of one or more L-amino acid residues with one or more D-amino acid residues. This embodiment is believed to increase proteolytic stability by steric hindrance and by a propensity of D-amino acids to stabilise □-turn conformations (Tugyi et al (2005) PNAS, 102(2), 413-418).

In one embodiment, the modified derivative comprises removal of any amino acid residues and substitution with alanines. This embodiment provides the advantage of removing potential proteolytic attack site(s).

It should be noted that each of the above mentioned modifications serve to deliberately improve the potency or stability of the peptide. Further potency improvements based on modifications may be achieved through the following mechanisms:
Incorporating hydrophobic moieties that exploit the hydrophobic effect and lead to lower off rates, such that higher affinities are achieved;
Incorporating charged groups that exploit long-range ionic interactions, leading to faster on rates and to higher affinities (see for example Schreiber et al, Rapid, electrostatically assisted association of proteins (1996), Nature Struct. Biol. 3, 427-31); and
Incorporating additional constraint into the peptide, by for example constraining side chains of amino acids correctly such that loss in entropy is minimal upon target binding, constraining the torsional angles of the backbone such that loss in entropy is minimal upon target binding and introducing additional cyclisations in the molecule for identical reasons.

(for reviews see Gentilucci et al, Curr. Pharmaceutical Design, (2010), 16, 3185-203, and Nestor et al, Curr. Medicinal Chem (2009), 16, 4399-418).

### Isotopic Variations

The present invention includes all pharmaceutically acceptable (radio)isotope-labeled peptide ligands of the invention, wherein one or more atoms are replaced by atoms having the same atomic number, but an atomic mass or mass number different from the atomic mass or mass number usually found in nature, and peptide ligands of the invention, wherein metal chelating groups are attached (termed “effector”) that are capable of holding relevant (radio)isotopes, and peptide ligands of the invention, wherein certain functional groups are covalently replaced with relevant (radio)isotopes or isotopically labelled functional groups.

Examples of isotopes suitable for inclusion in the peptide ligands of the invention comprise isotopes of hydrogen, such as ²H (D) and ³H (T), carbon, such as ¹¹C, ¹³C and ¹⁴C, chlorine, such as ³⁶Cl, fluorine, such as ¹⁸F, iodine, such as ¹²³I, ¹²⁵I and ¹³¹I, nitrogen, such as ¹³N and ¹⁵N, oxygen, such as ¹⁵O, ¹⁷O and ¹⁸O, phosphorus, such as ³²P, sulfur, such as ³⁵S, copper, such as ⁶⁴Cu, gallium, such as ⁶⁷Ga or ⁶⁸Ga, yttrium, such as ⁹⁰Y and lutetium, such as ¹⁷⁷Lu, and Bismuth, such as ²¹³Bi.

Certain isotopically-labelled peptide ligands of the invention, for example, those incorporating a radioactive isotope, are useful in drug and/or substrate tissue distribution studies. The peptide ligands of the invention can further have valuable diagnostic properties in that they can be used for detecting or identifying the formation of a complex between a labelled compound and other molecules, peptides, proteins, enzymes or receptors. The detecting or identifying methods can use compounds that are labelled with labelling agents such as radioisotopes, enzymes, fluorescent substances, luminous substances (for example, luminol, luminol derivatives, luciferin, aequorin and luciferase), etc. The radioactive isotopes tritium, i.e. ³H (T), and carbon-14, i.e. ¹⁴C, are particularly useful for this purpose in view of their ease of incorporation and ready means of detection.

Substitution with heavier isotopes such as deuterium, i.e. ²H (D), may afford certain therapeutic advantages resulting from greater metabolic stability, for example, increased in vivo half-life or reduced dosage requirements, and hence may be preferred in some circumstances.

Substitution with positron emitting isotopes, such as ¹¹C, ¹⁸F, ¹⁵O and ¹³N, can be useful in Positron Emission Topography (PET) studies for examining target occupancy.

Isotopically-labeled compounds of peptide ligands of the invention can generally be prepared by conventional techniques known to those skilled in the art or by processes analogous to those described in the accompanying Examples using an appropriate isotopically-labeled reagent in place of the non-labeled reagent previously employed.

### Molecular Scaffold

Molecular scaffolds are described in, for example, WO 2009/098450 and references cited therein, particularly WO 2004/077062 and WO 2006/078161.

As noted in the foregoing documents, the molecular scaffold may be a small molecule, such as a small organic molecule.

In one embodiment the molecular scaffold may be a macromolecule. In one embodiment the molecular scaffold is a macromolecule composed of amino acids, nucleotides or carbohydrates.

In one embodiment the molecular scaffold comprises reactive groups that are capable of reacting with functional group(s) of the polypeptide to form covalent bonds.

The molecular scaffold may comprise chemical groups which form the linkage with a peptide, such as amines, thiols, alcohols, ketones, aldehydes, nitriles, carboxylic acids, esters, alkenes, alkynes, azides, anhydrides, succinimides, maleimides, alkyl halides and acyl halides.

The molecular scaffold of the invention contains chemical groups that allow functional groups of the polypeptide of the encoded library of the invention to form covalent links with the molecular scaffold. Said chemical groups are selected from a wide range of functionalities including amines, thiols, alcohols, ketones, aldehydes, nitriles, carboxylic acids, esters, alkenes, alkynes, anhydrides, succinimides, maleimides, azides, alkyl halides and acyl halides.

Scaffold reactive groups that could be used on the molecular scaffold to react with thiol groups of cysteines are alkyl halides (or also named halogenoalkanes or haloalkanes).

Examples include bromomethylbenzene or iodoacetamide. Other scaffold reactive groups that are used to selectively couple compounds to cysteines in proteins are maleimides, □□ unsaturated carbonyl containing compounds and □□halomethylcarbonyl containing compounds. Examples of maleimides which may be used as molecular scaffolds in the invention include: tris-(2-maleimidoethyl)amine, tris-(2-maleimidoethyl)benzene, tris-(maleimido)benzene.

In one embodiment, the molecular scaffold is selected from 1,1′,1″-(1,3,5-triazinane-1,3,5-triyl)triprop-2-en-1-one (also known as triacryloylhexahydro-s-triazine; TATA), 1,3,5-tris(bromoacetyl) hexahydro-1,3,5-triazine (TATB) and 2,4,6-tris(bromomethyl)-s-triazine (TBMT).

In a further embodiment, the molecular scaffold is 1,1′,1″-(1,3,5-triazinane-1,3,5-tril)triprop-2-en-1-one (also known as triacryloylhexahydro-s-triazine (TATA):

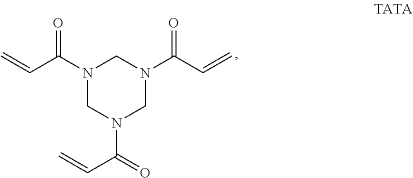 [SMILES: C=CC(=O)N1CN(C(=O)C=C)CN(C(=O)C=C)C1]

Thus, following cyclisation with the bicyclic peptides of the invention on the Cᵢ, Cᵢᵢ, and Cᵢᵢᵢ cysteine residues, the molecular scaffold forms a tri-substituted 1,1′,1″-(1,3,5-triazinane-1,3,5-triyl)tripropan-1-one derivative of TATA having the following structure:

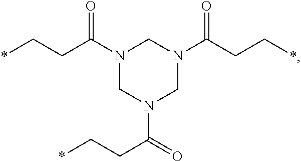

wherein * denotes the point of attachment of the three cysteine residues.

In an alternative embodiment, the molecular scaffold is 1,3,5-tris(bromoacetyl) hexahydro-1, 3,5-triazine (TATB):

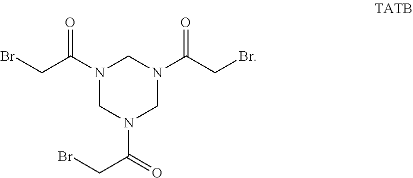 [SMILES: O=C(CBr)N1CN(C(=O)CBr)CN(C(=O)CBr)C1]

Thus, following cyclisation with the bicyclic peptides of the invention on the Cᵢ, Cᵢᵢ, and Cᵢᵢᵢ cysteine residues, the molecular scaffold forms a tri-substituted 1,3,5-tris(bromoacetyl) hexahydro-1,3,5-triazine derivative of TATB having the following structure:

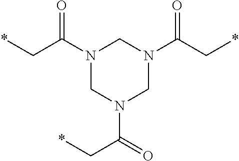 [SMILES: *CC(=O)N1CN(C(=O)C*)CN(C(=O)C*)C1]

wherein * denotes the point of attachment of the three cysteine residues.

In an alternative embodiment, the molecular scaffold is 2,4,6-tris(bromomethyl)-s-triazine (TBMT):

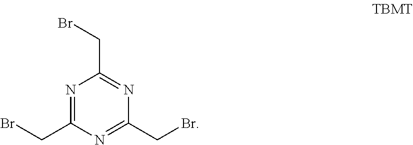 [SMILES: BrCc1nc(CBr)nc(CBr)n1]

Thus, following cyclisation with the bicyclic peptides of the invention on the Cᵢ, Cᵢᵢ, and Cᵢᵢᵢ, cysteine residues, the molecular scaffold forms a tri-substituted 2,4,6-tris(bromomethyl)-s-triazine derivative of TBMT having the following structure:

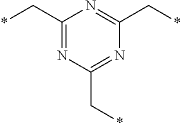 [SMILES: *Cc1nc(C*)nc(C*)n1]

wherein * denotes the point of attachment of the three cysteine residues.

Full details of TBMT and derivatisation are its use in cyclic peptides are described in van de Langemheen et al (2016) ChemBioChem 10.1002/cbic.201600612 (https://onlinelibrary.wiley.com/doi/abs/10.1002/cbic.201600612).

### Reactive Groups

The molecular scaffold of the invention may be bonded to the polypeptide via functional or reactive groups on the polypeptide. These are typically formed from the side chains of particular amino acids found in the polypeptide polymer. Such reactive groups may be a cysteine side chain, a [Dap(Me)] group, a lysine side chain, or an N-terminal amine group or any other suitable reactive group. Details may be found in WO 2009/098450. In one embodiment, the reactive groups are all cysteine residues.

Examples of reactive groups of natural amino acids are the thiol group of cysteine, the amino group of lysine, the carboxyl group of aspartate or glutamate, the guanidinium group of arginine, the phenolic group of tyrosine or the hydroxyl group of serine. Non-natural amino acids can provide a wide range of reactive groups including an azide, a keto-carbonyl, an alkyne, a vinyl, or an aryl halide group. The amino and carboxyl group of the termini of the polypeptide can also serve as reactive groups to form covalent bonds to a molecular scaffold/molecular core.

The polypeptides of the invention contain at least three reactive groups. Said polypeptides can also contain four or more reactive groups. The more reactive groups are used, the more loops can be formed in the molecular scaffold.

In a preferred embodiment, polypeptides with three reactive groups are generated. Reaction of said polypeptides with a molecular scaffold/molecular core having a three-fold rotational symmetry generates a single product isomer. The generation of a single product isomer is favourable for several reasons. The nucleic acids of the compound libraries encode only the primary sequences of the polypeptide but not the isomeric state of the molecules that are formed upon reaction of the polypeptide with the molecular core. If only one product isomer can be formed, the assignment of the nucleic acid to the product isomer is clearly defined. If multiple product isomers are formed, the nucleic acid cannot give information about the nature of the product isomer that was isolated in a screening or selection process. The formation of a single product isomer is also advantageous if a specific member of a library of the invention is synthesized. In this case, the chemical reaction of the polypeptide with the molecular scaffold yields a single product isomer rather than a mixture of isomers.

In another embodiment of the invention, polypeptides with four reactive groups are generated. Reaction of said polypeptides with a molecular scaffold/molecular core having a tetrahedral symmetry generates two product isomers. Even though the two different product isomers are encoded by one and the same nucleic acid, the isomeric nature of the isolated isomer can be determined by chemically synthesizing both isomers, separating the two isomers and testing both isomers for binding to a target ligand.

In one embodiment of the invention, at least one of the reactive groups of the polypeptides is orthogonal to the remaining reactive groups. The use of orthogonal reactive groups allows the directing of said orthogonal reactive groups to specific sites of the molecular core. Linking strategies involving orthogonal reactive groups may be used to limit the number of product isomers formed. In other words, by choosing distinct or different reactive groups for one or more of the at least three bonds to those chosen for the remainder of the at least three bonds, a particular order of bonding or directing of specific reactive groups of the polypeptide to specific positions on the molecular scaffold may be usefully achieved.

In another embodiment, the reactive groups of the polypeptide of the invention are reacted with molecular linkers wherein said linkers are capable to react with a molecular scaffold so that the linker will intervene between the molecular scaffold and the polypeptide in the final bonded state.

In some embodiments, amino acids of the members of the libraries or sets of polypeptides can be replaced by any natural or non-natural amino acid. Excluded from these exchangeable amino acids are the ones harbouring functional groups for cross-linking the polypeptides to a molecular core, such that the loop sequences alone are exchangeable. The exchangeable polypeptide sequences have either random sequences, constant sequences or sequences with random and constant amino acids. The amino acids with reactive groups are either located in defined positions within the polypeptide, since the position of these amino acids determines loop size.

In one embodiment, an polypeptide with three reactive groups has the sequence (X)ₗY(X)ₘY(X)ₙY(X)ₒ, wherein Y represents an amino acid with a reactive group, X represents a random amino acid, m and n are numbers between 3 and 6 defining the length of intervening polypeptide segments, which may be the same or different, and l and o are numbers between 0 and 20 defining the length of flanking polypeptide segments.

Alternatives to thiol-mediated conjugations can be used to attach the molecular scaffold to the peptide via covalent interactions. Alternatively these techniques may be used in modification or attachment of further moieties (such as small molecules of interest which are distinct from the molecular scaffold) to the polypeptide after they have been selected or isolated according to the present invention—in this embodiment then clearly the attachment need not be covalent and may embrace non-covalent attachment. These methods may be used instead of (or in combination with) the thiol mediated methods by producing phage that display proteins and peptides bearing unnatural amino acids with the requisite chemical reactive groups, in combination small molecules that bear the complementary reactive group, or by incorporating the unnatural amino acids into a chemically or recombinantly synthesised polypeptide when the molecule is being made after the selection/isolation phase. Further details can be found in WO 2009/098450 or Heinis, et al., Nat Chem Biol 2009, 5 (7), 502-7.

### Synthesis

The peptides of the present invention may be manufactured synthetically by standard techniques followed by reaction with a molecular scaffold in vitro. When this is performed, standard chemistry may be used. This enables the rapid large scale preparation of soluble material for further downstream experiments or validation. Such methods could be accomplished using conventional chemistry such as that disclosed in Timmerman et al. (supra).

Thus, the invention also relates to manufacture of polypeptides selected as set out herein, wherein the manufacture comprises optional further steps as explained below. In one embodiment, these steps are carried out on the end product polypeptide made by chemical synthesis.

Peptides can also be extended, to incorporate for example another loop and therefore introduce multiple specificities.

To extend the peptide, it may simply be extended chemically at its N-terminus or C-terminus or within the loops using orthogonally protected lysines (and analogues) using standard solid phase or solution phase chemistry. Standard (bio)conjugation techniques may be used to introduce an activated or activatable N- or C-terminus. Alternatively, additions may be made by fragment condensation or native chemical ligation e.g. as described in (Dawson et al. 1994. Synthesis of Proteins by Native Chemical Ligation. Science 266:776-779), or by enzymes, for example using subtiligase as described in (Chang et al. Proc Natl Acad Sci USA. 1994 Dec 20; 91(26):12544-8 or in Hikari et al Bioorganic & Medicinal Chemistry Letters Volume 18, Issue 22, 15 Nov. 2008, Pages 6000-6003).

Alternatively, the peptides may be extended or modified by further conjugation through disulphide bonds. This has the additional advantage of allowing the first and second peptide to dissociate from each other once within the reducing environment of the cell. In this case, the molecular scaffold (e.g. TATA, TATB or TBMT) could be added during the chemical synthesis of the first peptide so as to react with the three cysteine groups; a further cysteine or thiol could then be appended to the N or C-terminus of the first peptide, so that this cysteine or thiol only reacted with a free cysteine or thiol of the second peptide, forming a disulfide—linked bicyclic peptide-peptide conjugate.

Similar techniques apply equally to the synthesis/coupling of two bicyclic and bispecific macrocycles, potentially creating a tetraspecific molecule.

Furthermore, addition of other functional groups or effector groups may be accomplished in the same manner, using appropriate chemistry, coupling at the N- or C-termini or via side chains. In one embodiment, the coupling is conducted in such a manner that it does not block the activity of either entity.

The multimeric complexes of the invention may be prepared in accordance with analogous methodology to that described in WO 2019/162682.

### Synthesis of BCY18656

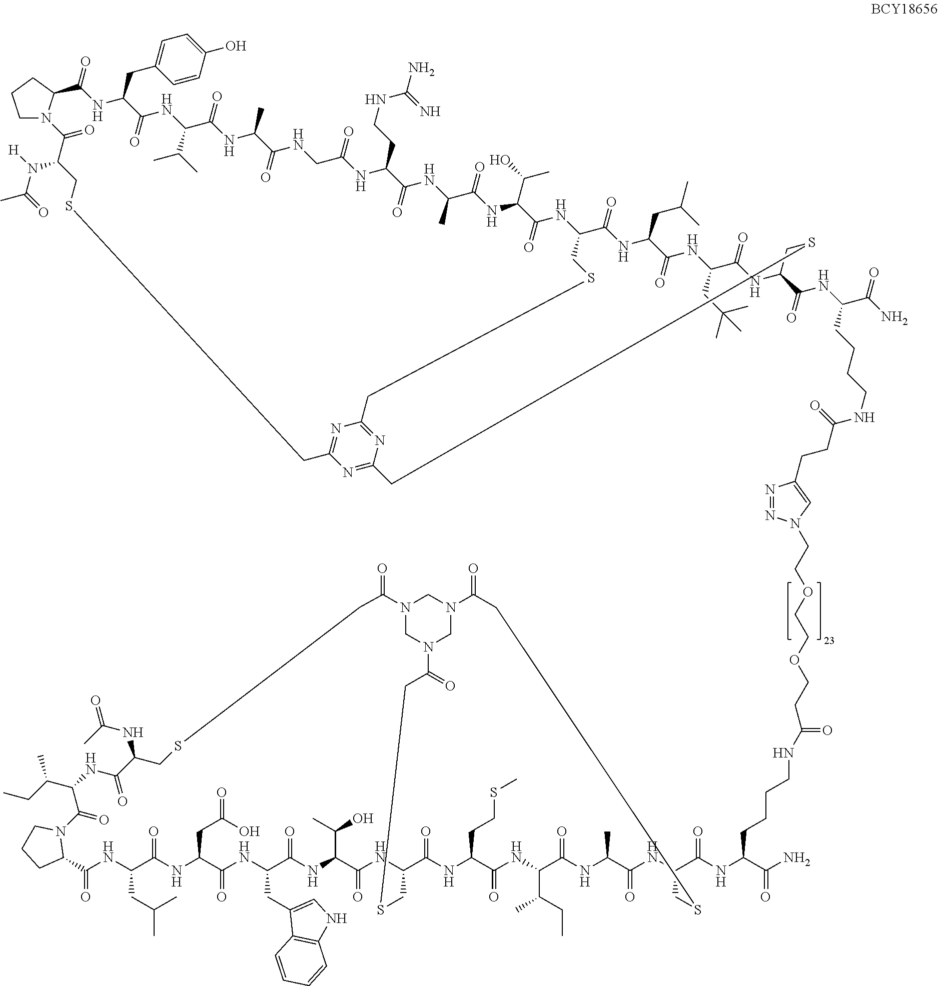

### Preparation of Intermediate BCY18655

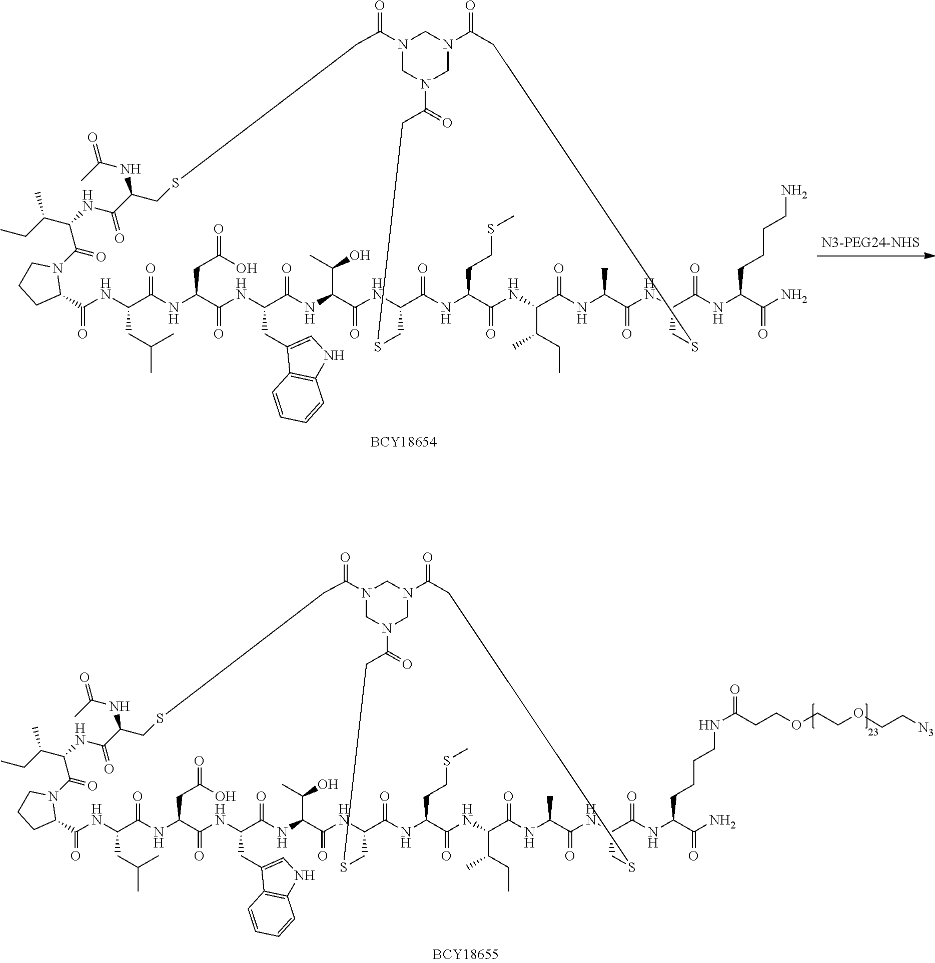

A mixture of BCY18654 (20.0 mg, 11.46 μmol, 1.0 eq.), and N3-PEG24-NHS (17.5 mg, 13.75 μmol, 1.2 eq.) was dissolved in DMF (0.5 mL), then DIEA (4.0 μL, 22.92 μmol, 2.0 eq.) was added into the mixture. The reaction mixture was stirred at 25-30° C. for 0.5 hr. LC-MS showed compound 1 was consumed completely and one main peak with desired m/z (MW: 2899.45, observed m/z: 1450.3 ([(M/2+H]+), 967.3 ([(M/3+H]+)) was detected. The reaction mixture was filtered to remove the undissolved residue. The crude product was then purified by prep-HPLC BCY18655 (17.2 mg, 5.778 μmol, 50.31% yield, 97.2% purity) was obtained as a white solid.

### Preparation of Final Product BCY18656

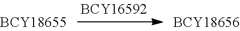

A mixture of BCY18655 (17.2 mg, 5.93 μmol, 1.0 eq., prepared as described above), BCY16592 (11.3 mg, 6.53 μmol, 1.1 eq.), and THPTA (2.6 mg, 5.93 μmol, 1.0 eq.) was dissolved in t-BuOH/H₂O (1:1, 0.5 mL, pre-degassed and purged with N₂ for 3 times), and then aqueous solution of CuSO₄ (0.4 M, 14.8 μl, 1.0 eq.) and VcNa (2.3 mg, 11.86 μmol, 2.0 eq.) were added under N₂. The pH of this solution was adjusted to 8 by dropwise addition of 0.2 M NH₄HCO₃ (in 1:1 t-BuOH/H₂O), and the solution turned to light yellow. The reaction mixture was stirred at 25-30° C. for 0.5 hr under N₂ atmosphere. LC-MS showed BCY18655 was consumed completely, and one main peak with desired m/z (calculated MW: 4635.53, observed m/z: 1159.7 ([M/4+H]+), 924.5 ([M/5+H]+), M-18 (m/z=1155.4 and 924.5) was also observed) was detected. The reaction mixture was filtered to remove the undissolved residue. The crude product was purified by prep-HPLC and BCY18656 (15.6 mg, 3.25 μmol, 54.80% yield, 96.6% purity) was obtained as a white solid.

### Pharmaceutical Compositions

According to a further aspect of the invention, there is provided a pharmaceutical composition comprising a peptide ligand as defined herein in combination with one or more pharmaceutically acceptable excipients.

Generally, the present peptide ligands will be utilised in purified form together with pharmacologically appropriate excipients or carriers. Typically, these excipients or carriers include aqueous or alcoholic/aqueous solutions, emulsions or suspensions, including saline and/or buffered media. Parenteral vehicles include sodium chloride solution, Ringer's dextrose, dextrose and sodium chloride and lactated Ringer's. Suitable physiologically-acceptable adjuvants, if necessary to keep a polypeptide complex in suspension, may be chosen from thickeners such as carboxymethylcellulose, polyvinylpyrrolidone, gelatin and alginates.

Intravenous vehicles include fluid and nutrient replenishers and electrolyte replenishers, such as those based on Ringer's dextrose. Preservatives and other additives, such as antimicrobials, antioxidants, chelating agents and inert gases, may also be present (Mack (1982) Remington's Pharmaceutical Sciences, 16th Edition).

The compounds of the invention can be used alone or in combination with another agent or agents.

The compounds of the invention can also be used in combination with biological therapies such as nucleic acid based therapies, antibodies, bacteriophage or phage lysins.

The route of administration of pharmaceutical compositions according to the invention may be any of those commonly known to those of ordinary skill in the art. For therapy, the peptide ligands of the invention can be administered to any patient in accordance with standard techniques. Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly. Preferably, the pharmaceutical compositions according to the invention will be administered parenterally. The dosage and frequency of administration will depend on the age, sex and condition of the patient, concurrent administration of other drugs, counterindications and other parameters to be taken into account by the clinician.

The peptide ligands of this invention can be lyophilised for storage and reconstituted in a suitable carrier prior to use. This technique has been shown to be effective and art-known lyophilisation and reconstitution techniques can be employed. It will be appreciated by those skilled in the art that lyophilisation and reconstitution can lead to varying degrees of activity loss and that levels may have to be adjusted upward to compensate.

The compositions containing the present peptide ligands or a cocktail thereof can be administered for therapeutic treatments. In certain therapeutic applications, an adequate amount to accomplish at least partial inhibition, suppression, modulation, killing, or some other measurable parameter, of a population of selected cells is defined as a “therapeutically-effective dose”. Amounts needed to achieve this dosage will depend upon the severity of the disease and the general state of the patient's own immune system, but generally range from 10 μg to 250 mg of selected peptide ligand per kilogram of body weight, with doses of between 100 μg to 25 mg/kg/dose being more commonly used.

A composition containing a peptide ligand according to the present invention may be utilised in therapeutic settings to treat a microbial infection or to provide prophylaxis to a subject at risk of infection e.g. undergoing surgery, chemotherapy, artificial ventilation or other condition or planned intervention. In addition, the peptide ligands described herein may be used extracorporeally or in vitro selectively to kill, deplete or otherwise effectively remove a target cell population from a heterogeneous collection of cells. Blood from a mammal may be combined extracorporeally with the selected peptide ligands whereby the undesired cells are killed or otherwise removed from the blood for return to the mammal in accordance with standard techniques.

### Therapeutic Uses

The bicyclic peptides of the invention have specific utility as severe acute respiratory syndrome coronavirus 2 (SARS-CoV-2) binding agents.

Polypeptide ligands selected according to the method of the present invention may be employed in in vivo therapeutic applications, in vitro and in vivo diagnostic applications, in vitro assay and reagent applications, and the like. In some applications, such as vaccine applications, the ability to elicit an immune response to predetermined ranges of antigens can be exploited to tailor a vaccine to specific diseases and pathogens.

Substantially pure peptide ligands of at least 90 to 95% homogeneity are preferred for administration to a mammal, and 98 to 99% or more homogeneity is most preferred for pharmaceutical uses, especially when the mammal is a human. Once purified, partially or to homogeneity as desired, the selected polypeptides may be used diagnostically or therapeutically (including extracorporeally) or in developing and performing assay procedures, immunofluorescent stainings and the like (Lefkovite and Pernis, (1979 and 1981) Immunological Methods, Volumes I and II, Academic Press, NY).

According to a further aspect of the invention, there is provided a peptide ligand as defined herein, for use in suppressing or treating a disease or disorder mediated by infection of SARS-CoV-2 or for providing prophylaxis to a subject at risk of infection of SARS-CoV-2.

According to a further aspect of the invention, there is provided a method of suppressing or treating a disease or disorder mediated by infection of SARS-CoV-2 or for providing prophylaxis to a subject at risk of infection of SARS-CoV-2, which comprises administering to a patient in need thereof the peptide ligand as defined herein.

References herein to “disease or disorder mediated by infection of SARS-CoV-2” include: respiratory disorders, such as a respiratory disorder mediated by an inflammatory response within the lung, in particular COVID-19.

References herein to the term “suppression” refers to administration of the composition after an inductive event, but prior to the clinical appearance of the disease. “Treatment” involves administration of the protective composition after disease symptoms become manifest.

Animal model systems which can be used to screen the effectiveness of the peptide ligands in protecting against or treating the disease are available.

### Screening Methods

It will be appreciated that the multimeric binding complexes of the invention also find utility as agents for screening for other SARS-CoV-2 binding agents.

For example, screening for a SARS-CoV-2 binding agent may typically involve incubating a multimeric binding complex of the invention with SARS-CoV-2 in the presence and absence of a test compound and assessing a difference in the degree of binding, such that a difference in binding will result from competition of the test compound with the multimeric binding complex of the invention for binding to SARS-CoV-2.

Thus, according to a further aspect of the invention, there is provided a method of screening for a compound which binds to SARS-CoV-2 wherein said method comprises the following steps:
(a) incubating a multimeric binding complex as defined herein with SARS-CoV-2;
(b) measuring the binding activity of said multimeric binding complex;
(c) incubating said multimeric binding complex from step (a) with a test compound and SARS-CoV-2;
(d) measuring the binding activity of said multimeric binding complex; and
(e) comparing the binding activity in steps (b) and (d), such that a difference in binding activity of said multimeric binding complex is indicative of the test compound binding to SARS-CoV-2.

In one embodiment, the multimeric binding complex comprises a reporter moiety for ease of detecting binding. In a further embodiment, the reporter moiety comprises fluorescein (Fl). In a yet further embodiment, the multimeric binding complex comprises any of the peptide ligands described herein which additionally comprise a fluorescein (Fl) moiety.

### Diagnostic Methods

It will be appreciated that the bicyclic peptide ligands of the invention also find utility as agents for diagnosing infection of SARS-CoV-2.

For example, diagnosis of SARS-CoV-2 infection may typically involve incubating a multimeric binding complex of the invention with SARS-CoV-2 in the presence and absence of a test compound and assessing a difference in the degree of binding, such that a difference in binding will result from competition of the test compound with the multimeric binding complex of the invention for binding to SARS-CoV-2.

Thus, according to a further aspect of the invention, there is provided a method of diagnosing SARS-CoV-2 infection wherein said method comprises the following steps:
a) obtaining a biological sample from an individual;
(b) incubating a multimeric binding complex as defined herein with the biological sample obtained in step (a); and
(c) detecting binding of said multimeric binding complex to SARS-CoV-2, such that a detection of measurable binding activity is indicative of a diagnosis of SARS-CoV-2 infection.

In one embodiment, the peptide ligand comprises a reporter moiety for ease of detecting binding. In a further embodiment, the reporter moiety comprises fluorescein (Fl). In a yet further embodiment, the multimeric binding complex comprises any of the peptide ligands described herein which additionally comprise a fluorescein (Fl) moiety.

The invention is further described below with reference to the following examples.

### EXAMPLES

### Materials and Methods

### Peptide Synthesis

Peptide synthesis was based on Fmoc chemistry, using a Symphony peptide synthesiser manufactured by Peptide Instruments and a Syro II synthesiser by MultiSynTech. Standard Fmoc-amino acids were employed (Sigma, Merck), with appropriate side chain protecting groups: where applicable standard coupling conditions were used in each case, followed by deprotection using standard methodology.

Alternatively, peptides were purified using HPLC and following isolation they were modified with the required molecular scaffold (namely, TATA, TATB or TBMT). For this, linear peptide was diluted with 50:50 MeCN:H₂O up to ˜35 mL, ˜500 μL of 100 mM scaffold in acetonitrile was added, and the reaction was initiated with 5 mL of 1 M NH₄HCO₃ in H₂O. The reaction was allowed to proceed for ˜30−60 min at RT, and lyophilised once the reaction had completed (judged by MALDI). Once completed, 1 ml of 1M L-cysteine hydrochloride monohydrate (Sigma) in H₂O was added to the reaction for ˜60 min at RT to quench any excess TATA, TATB or TBMT.

Following lyophilisation, the modified peptide was purified as above, while replacing the Luna C8 with a Gemini C18 column (Phenomenex), and changing the acid to 0.1% trifluoroacetic acid. Pure fractions containing the correct scaffold-modified material were pooled, lyophilised and kept at −20° C. for storage.

All amino acids, unless noted otherwise, were used in the L-configurations.

In some cases peptides are converted to activated disulfides prior to coupling with the free thiol group of a toxin using the following method; a solution of 4-methyl(succinimidyl 4-(2-pyridylthio)pentanoate) (100 mM) in dry DMSO (1.25 mol equiv) was added to a solution of peptide (20 mM) in dry DMSO (1 mol equiv). The reaction was well mixed and DIPEA (20 mol equiv) was added. The reaction was monitored by LC/MS until complete.

### Mutimeric Binding Complex Synthesis

The multimeric complexes of the invention may be prepared in accordance with analogous methodology to that described in WO 2019/162682.

### BIOLOGICAL DATA

### 1. Pseudoviral Neutralisation Assay

Replication deficient SARS-CoV-2 pseudotyped HIV-1 virions were prepared similarly as described in Mallery et al (2021) Sci Adv 7(11). Briefly, virions were produced in HEK 293T cells by transfection with 1 μg of the plasmid encoding SARS CoV-2 Spike protein (pCAGGS-SpikeΔc19), 1 μg pCRV GagPol and 1.5 μg GFP-encoding plasmid (CSGVV). Viral supernatants were filtered through a 0.45 μm syringe filter at 48 h and 72 h post-transfection and pelleted for 2 h at 28,000×g. Pelleted virions were drained and then resuspended in DMEM (Gibco).

HEK 293T-hACE2-TMPRSS2 cells were prepared as described in Papa et al (2021) PLoS Pathog. 17(1), p. e1009246. Cells were plated into 96-well plates at a density of 2×103 cells per well in Free style 293T expression media and allowed to attach overnight. 18 μl pseudovirus-containing supernatant was mixed with 2 μl dilutions of bicycle peptide and incubated for 40 min at RT. 10 μl of this mixture was added to cells. 72 h later, cell entry was detected through the expression of GFP by visualisation on an Incucyte S3 live cell imaging system (Sartorius). The percent of cell entry was quantified as GFP positive areas of cells over the total area covered by cells. Entry inhibition by the Bicyclic peptide was calculated as percent virus infection relative to virus only control.

Certain multimeric binding complexes of the invention were tested in the above assay and the results shown in Table 1:

TABLE 1
Pseudoviral Neutralisation Assay Results for Selected
Multimeric Binding Complexes of the Invention

| Multimeric | Geomean | Pseudovirus |
|---|---|---|
| Binding Complex | IC₅₀ (nM) | Construct |
| BCY17187 | 144 | |
| BCY17188 | 31100 | |
| BCY17190 | 147 | |
| BCY17191 | 10.6 | |
| BCY17192 | 18 | |
| BCY17193 | 8.8 | |
| BCY17195 | 857 | |
| BCY17196 | 15300 | |
| BCY17197 | 33.9 | |
| BCY17198 | 0.0289 | |
| BCY17199 | 5.46 | |
| BCY18653 | 905 | |
| BCY18786 | 0.2 | |
| BCY17198 | 0.3 | D614G |
| BCY18653 | 906 | |
| BCY18786 | 20 | |
| BCY17198 | 3 | B.1.1.7 (Alpha) |
| BCY18653 | 18 | |
| BCY18786 | 6 | |
| BCY18786 | 272 | B.1.351 (Beta) |
| BCY18786 | 140 | P.1 (Gamma) |
| BCY18786 | 437 | B.1.617.2 (Delta) |
| BCY18786 | 93 | B.1.427 (California) |

### 2. SARS-CoV-2 Cytopathic Effect (CPE)

A549_ACE_TMPRSS2 cells were seeded in 96-well plates and cultured overnight. The following day, 4-fold serial dilutions of the bicycle compounds were prepared in medium and 60 μl of the diluted compounds starting from a maximum concentration of 30, 15, 10, 3, 1, or 0.1 μM were added to the plates with cells. After 3 h pre-incubation, cells were infected with SARS-CoV-2 GL
A-1 at MOI 0.04 PFU/cell. One dose of 522 PFU of the virus in 60 μl per well was added to the wells containing compounds. Plates were incubated for 72 h at 37° C., fixed and stained when the cytopathic effect (CPE) was visible. Plates were scanned in a plate reader to quantitate the levels of CPE.

Certain multimeric binding complexes of the invention were tested in the above assay and the results shown in Table 2:

TABLE 2
Cytopathic Effect Assay Results for Selected
Multimeric Binding Complexes of the Invention

| Multimeric | IC₅₀ |
|---|---|
| Binding Complex | (nM) |
| BCY17188 | 21400 |
| BCY17190 | 10600 |
| BCY17192 | 9330 |
| BCY17193 | 19400 |
| BCY17198 | 0.303 |
| BCY17199 | 898 |
| BCY18653 | 159 |
| BCY18656 | 0.314 |
| BCY18786 | 246 |

### 3. Hamster in vivo SARS-CoV-2 Challenge Study with BCY18656

Different animal models for studying SARS-CoV-2 have been tested and include Syrian hamsters, ferrets and rabbits. In this study, the hamster model was used to investigate the efficacy of SARS-CoV-2 specific antiviral compounds against challenge with SARS-CoV-2 as this model shows clinical signs (body weight loss), viral replication in both the upper and lower respiratory tract and histopathological changes.

To evaluate the protective efficacy of BCY18656 against SARS-CoV-2 in the hamster model the following setup was used. Animals were randomly assigned to one of 5 groups with 5 animals per group and treated with BCY18656 at 10, 3 or 1 mg/kg (group 1-3), or vehicle (group 5). The vehicle will consist of the sucrose histidine buffer that was used to reconstitute and dilute the compounds. Treatment was started 4 hours by the subcutaneous route in the neck with a volume of 200 μl/100 gram before challenge after which the animals were challenged intranasally (i.n.) with 10² TCID₅₀ SARS-CoV-2 in total dose volume of 100 μl divided equally over both nostrils. Subsequently, treatment was continued with an interval of 8 hours up to and including day 3 post challenge (p.c.). Animals were weighed and monitored daily and swabs were taken pre-infection (day 0) and then daily p.c. On day 4 p.c., animals were euthanised for sampling for virology and (histo)pathology.

The in vivo phase of this study was conducted by Viroclinics Biosciences B.V., Viroclinics Xplore in their animal facility in Schaijk, The Netherlands. Management, coordination, sample processing, serological and virological analyses, and interpretation of the data was conducted by Viroclinics Biosciences B.V., Viroclinics Xplore, Schaijk, The Netherlands. Gross pathology was performed by a board-certified veterinary pathologist.

### Objective of the Study

The goal of the current study was to investigate the prophylactic efficacy of BCY18656 against SARS-CoV-2 challenge in the hamster model.

### Test Materials

### Test Items

| | |
|---|---|
| Full description | BCY18656 |
| Lot number | N0949-12-1 |
| Expiry date | Not assigned |
| Dosage | 10, 3 and 1 mg/kg |
| Storage | Room temperature |
| conditions | |
| Route of | Subcutaneous |
| administration | |
| Volume of | 200 μl/100 g body weight |
| administration | |
| Concentration for | 5, 1.5 and 0.5 mg/ml |
| administration | |
| Formulation | As defined below |

### Formulation of Test Items

### Vehicle Preparation

Preparation of 1 L of buffer:
NaOH 1 M: 4 g was dissolved in 100 ml of water. NaOH 1 M was prepared to neutralize the 25 mM His*HCl solution with 10% of sucrose
5.24 g of His*HCl [209.63] was dissolved in 450 ml of water (55.5 mM His*HCl). The solution needed to be sonicated and stirred.
100 g of sucrose was separately dissolved in 450 ml of water (22.2% of sucrose). The solution needed to be sonicated and stirred.
His*HCl solution and sucrose solution were mixed together.
Sequential aliquots of NaOH 1M were added to reach the desired pH 7 as shown in the following table:

| | | |
|---|---|---|
| Solution | NaOH 1M added | pH of the solution |

| | | | |
|---|---|---|---|
| 900 mL 27.8 | | | 3.8 |
| mM His*HCl, | +10 | ml | 5.8 |
| 11.1% sucrose | +5 | ml | 6.2 |
| | +2.5 | ml | 6.4 |
| | +1 | ml | 6.5 |
| | +2 | ml | 6.7 |
| | +2 | ml | 6.95 |
| | +0.25 | ml | 7.00 |

Following this table 22.75 ml of NaOH 1M was added to 900 ml His*HCl and sucrose to neutralize the solution at pH 7.
77.25 ml of water was added to aim for 1 L of 25 mM His*HCl, 10% sucrose pH 7.
The final solution was filtered with 0.2 μM filters and stored at +4° C. during the in life phase of the study.

### Formulation of Test Items

Solutions of the compounds at the concentration defined in the table above (mg/mL) were prepared in 25 mM His*HCl, 10% sucrose pH 7 neutralised with NaOH, clear solution. Fresh dosing solutions for each compound and each concentration were prepared on each day of the experiment for three administration time-points at a total volume of 10 ml. After preparation of the solutions these were stored at 4° C. for a maximum period of 24 hours.

### Infection Material

| | |
|---|---|
| Full description | SARS-CoV-2 |
| Strain | BetaCoV/Munich/BavPat1/2020 |
| Origin | Vero E6 Cell culture |
| Passage | P3 on Vero-E6 |
| Lot number | VC-200180004 |
| Concentration | 7.1 log₁₀ TCID₅₀/ml |
| Manufacturing date | 17 Feb. 2020 |
| Expiry date | Not applicable* |
| Presentation | frozen liquid |
| storage condition | −70° C. or colder |
| (bio-) safety classification | Class III |

*stock was titrated on a regular basis to confirm the infectious titer.

### Infection Material Formulation

| | |
|---|---|
| Full description | SARS-CoV-2 |
| dosage for administration | 10\hat{ }2 TCID₅₀ |
| (bio-) safety classification | class III |
| formulation for | Virus was diluted prior to infection |
| administration | with cold PBS |
| expiry time | unknown (virus dilution was used |
| | within 2 hours after preparation). |
| storage condition before | until administration the challenge |
| administration | virus dilution was kept at 4° C. |
| route of administration | i.n. |
| administration volume | 100 μl |
| Vehicle | PBS |

### Test System

### Description of Test System

In vivo Syrian hamsters (see table below).

### Animal Husbandry

### Animal Housing

The animals were housed according to SOP VCX-P073 (Animal housing and welfare management) in elongated type 2 IVC group cages with two animals per cage under DM-2 conditions during acclimatization and in elongated type 2 group cages under DM-3 conditions (isolators) during challenge using sawdust as bedding. They were checked daily for overt signs of disease.

### Veterinary Care

The animal experiments were carried out in the central animal facilities of Viroclinics Xplore in Schaijk, The Netherlands, under conditions that meet the standard of Dutch law for animal experimentation (2010/63/EU) and are in agreement with the “Guide for the care and use of laboratory animals” (8^{th} edition, NRC 2011), ILAR recommendations, AAALAC standards. The facility is fully accredited by the Dutch ministry that governs and inspects the animal facilities and oversees, coordinates and inspects activities of the animal ethics committees of Dutch institutions and academic centres. An animal veterinarian of the test facility is in charge of animal welfare and medical care of animals in the test facility.

The Study Director is a registered article 9 (WoD) officer and responsible for the design of the animal experiments, in close consultation with the animal welfare body and the laboratory animal veterinarians. Ethics approval for the present study was registered under number: 27700202114492-WP16.

### Procedures to Limit Pain and Discomfort

Animals were evaluated daily for any adverse effects and complications. Animals were sedated for all procedures requiring handling and sampling, as described below. This is a standard procedure, with monitoring of sedated animals by trained animal technicians or veterinary technologist assigned to the area. Analgesic (buprenorphine or equivalent) were administered if recommended by the attending veterinarian. Animals exhibiting any pain or distress that cannot be controlled by anaesthetics or analgesics were removed from study and euthanized.

### Experimental Protocol

### Animals Required

| | |
|---|---|
| Species | Syrian hamster (Mesocricetus auratus) |
| Strain | RjHan:AURA (outbred) |
| Supplier | Janvier |
| Microbiological status | SPF |
| Number | 25 |
| Sex | Male |
| Age | ~10 weeks old at the start of the experiment |
| Body weight range | Variable, actual weight range was provided |
| | in the study report. |
| Identification | Animals were uniquely identified before start |
| | of the experiment with animal markers. |

### Animal Handling

### Anesthesia

For all animal procedures, the animals were sedated with isoflurane (3-4%/O₂) according to standard procedures known in the art. These procedures include subcutaneous and intranasal dosing, blood sampling, challenge, throat swabs and euthanasia.

### Blood Sampling

Before challenge on day 0-200 μl blood was collected for serum under isoflurane anesthesia. In short, the animal was scruffed with thumb and forefinger of the nondominant hand and the skin around the eye was pulled taut. A capillary was inserted into the medial canthus of the eye (30 degree angle to the nose). Slight pressure was applied to puncture the tissue and enter the plexus/sinus. Once the plexus/sinus was punctured, blood will come through the capillary tube. When the required volume of blood was collected from plexus, the capillary tube was gently removed and if applicable, bleeding can be stopped by applying gentle pressure. Blood samples for serum were immediately transferred to appropriate tubes containing a clot activator. Serum (˜100 μl) was collected stored at <−70° C.

### Subcutaneous Administration

For subcutaneous administration, the skin of the neck was grabbed with thumb and finger(s) to create a dimple. The needle (25G; 0.50×16 mm) was placed in the middle of the dimple between the fingers. The needle was injected as far as possible, to prevent the liquid flowing back. The needle was felt moving between the fingers to inject the correct volume of test item. Finally, the needle was removed in a smooth motion and the animal was placed back in their cage and monitored during recovery.

### Intranasal Administration

For intranasal administration the animals were held on their back and the inoculum (100 μl) was equally divided over both nostrils using an adjustable mono channel pipet. Animals were held on their back until the complete inoculum was inhaled after which they were placed back in the cage to recover.

### Clinical Observations

Observations were conducted and noted daily by the animal facility technicians, and daily following challenge by the laboratory technicians. These included ruffled fur, hunched back posture, accelerated breathing and lethargy and were noted down when observed.

Animals were weighed on regular time points during the study using electronic scales (internal individual scale number and performance were documented on appropriate forms). Body weight was recorded on appropriate forms. The performance of the scales was verified during the just before and after procedures using calibration weights, which were recorded on appropriate forms.

### Precautions

The precautions taken were that of handling of animals, manipulation of sharps and working under standard conditions.

### Sampling Post Inoculation

The respiratory tract was sampled on selected time points during the study. In short, throat swabs (FLOQSwabs, COPAN Diagnostic Inc., Italy) were used to sample the pharynx by rubbing the swabs against the back of the animal's throat saturating the swab with saliva. Subsequently, the swab was placed in a tube containing 1.5 ml virus transport medium (Eagles minimal essential medium containing Hepes buffer, Na bicarbonate solution, L-Glutamin, Penicillin, Streptomycin, BSA fraction V and Amphothericine B), aliquoted in three aliquots and stored.

### Tissue Collection at Necropsy

Upon necropsy, lung and nose tissue were collected and stored in 10% formalin for histopathology and immunohistochemistry and frozen for virological analysis. For virological analysis, lung and nose tissue samples were weighed, homogenized in 1.5 ml inoculation medium (DMEM containing L-Glutamin, Penicillin, Streptomycin, Amphothericin B and Fetal Bovine serum) and centrifuged briefly before titration.

### Pre-Study Protocol

### Blinding/Bias-Reducing Methods

All personnel performing the clinical observations and laboratory analysis in which interpretation of the data was required were not aware of the Random Treatment Allocation

Key at any time prior to completion of the study and were blinded by allocating a unique sample number to each sample collected.

### Study Protocol

### Treatment Protocol

All animals were administered on days 0 up to and including day 3. Animals were treated via the subcutaneous route.

### Challenge Protocol

On day 0, all animals were infected intranasally with SARS-CoV-2 (in a total dose volume of 100 μl). After infection an aliquot of the challenge virus dilution was stored at −80° C.

### Pathology

### Prematurely Euthanized or Dead Animals

When animals prematurely died or were prematurely sacrificed due to e.g. reaching humane end-points the above mentioned tissues were collected for virological and histopathological assessment.

### Gross-Pathology

At the time of necropsy for all animals (either found dead post infection, euthanised due to reaching humane endpoint or at experimental endpoint), gross pathology was performed on each animal and all abnormalities were described. All lung lobes were inspected, an estimation of the percentage affected lung tissue from the dorsal view were described, in addition, any other abnormalities observed in other organs during full body gross-pathology were also recorded.

Left lung lobes and nasal turbinates were preserved in 10% neutral buffered formalin for histopathology with the right side of these tissues subsequently homogenised and subjected to Taqman PCR and virus titration.

### Laboratory Investigations

### Description of Analyses

| Sample | Day(s) of | Responses | Method(s) of |
|---|---|---|---|
| type | sampling | analysed | analysis |
| 3. Throat | 4. Days 0 | 5. Replication | 8. Virus titration |
| swab | up to day 4 | competent | (SOP VC-M197) |
| | post inoculation | virus (TCID₅₀) | 9. |
| | | 6. | 10. Taqman (SOP |
| | | 7. Viral RNA | VC-M098 and -M052) |
| 11. Tissue | 12. Day | 13. Replication | 16. Virus |
| (lung and | of death | competent | titration |
| nasal | | virus (TCID₅₀) | (SOP VC-M197) |
| turbinates) | | 14. | 17. |
| | | 15. Viral RNA | 18. Taqman (SOP |
| | | | VC-M098 and -M052) |

### Virological Analyses

### Detection of Replication Competent Virus

Quadruplicate 10-fold serial dilutions were used to determine the virus titers in confluent layers of Vero E6 cells. To this end, serial dilutions of the samples (throat swabs and tissue homogenates) were made and incubated on Vero E6 monolayers for 1 hour at 37° C. Vero E6 monolayers were washed and incubated for 4-6 days at 37° C. after which plates were scored using the vitality marker WST8 (colorimetric readout). To this end, WST-8 stock solution was prepared and added to the plates. Per well, 20 μl of this solution (containing 4 μl of the ready-to-use WST-8 solution from the kit and 16 μ inoculation medium, 1:5 dilution) was added and incubated 3-5 hours at room temperature. Subsequently, plates were measured for optical density at 450 nm (OD450) using a micro plate reader and visual results of the positive controls (cytopathic effect (cpe)) were used to set the limits of the WST-8 staining (OD value associated with cpe). Viral titers (TCID₅₀) were calculated using the method of Spearman-Karber.

### Detection of Viral RNA

Throat swabs and homogenized tissue samples were used to detect viral RNA. To this end RNA was isolated and Taqman PCR was performed using specific primers:

E_Sarbeco_F:
(SEQ ID NO: 116)
ACAGGTACGTTAATAGTTAATAGCGT;
and
E_Sarbeco_R:
(SEQ ID NO: 117)
ATATTGCAGCAGTACGCACACA;
and
probe:
E_Sarbeco_P1:
(SEQ ID NO: 118)
ACACTAGCCATCCTTACTGCGCTTCG;

as described by Corman et al (https://doi.org/10.2807/1560-7917.ES.2020.25.3.2000045) with the TaqMan® Fast Virus 1-Step Master Mix (ThermoFischer Scientific). The number of virus copies in the different samples was calculated.

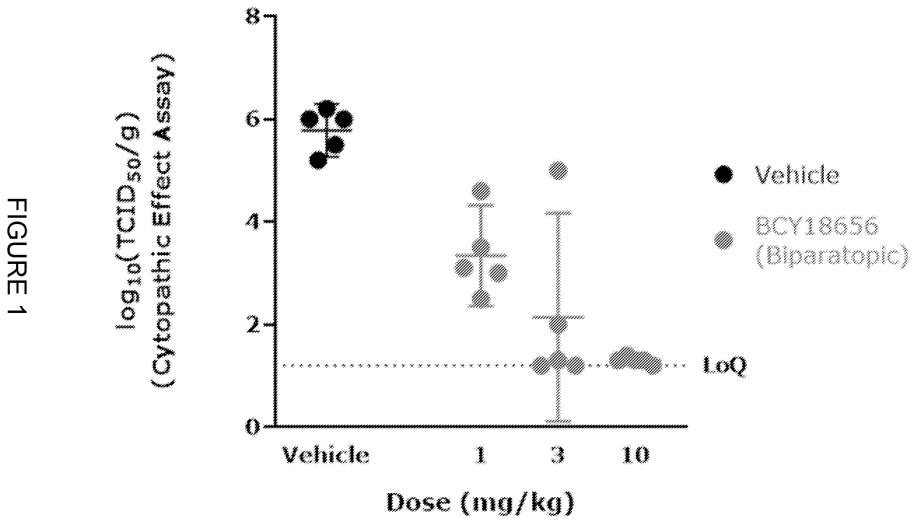

The results are shown in FIG. 1 where it can be seen that BCY18656 reduces the viral load of SARS-CoV-2 in hamsters after 3 days dosing at 1, 3 or 10 mg/kg compared to vehicle.

1. A multimeric binding complex which comprises at least two differing bicyclic peptide ligands, each of which comprises a peptide ligand specific for severe acute respiratory syndrome coronavirus 2 (SARS-CoV-2) comprising a polypeptide comprising at least three reactive groups, separated by at least two loop sequences, and a molecular scaffold which forms covalent bonds with the reactive groups of the polypeptide such that at least two polypeptide loops are formed on the molecular scaffold.

2. The multimeric binding complex according to claim 1, wherein said peptide ligand is specific for the spike protein (S protein) of SARS-CoV-2.

3. The multimeric binding complex according to claim 1 or claim 2, wherein said peptide ligand is specific for the S1 of S2 domain of the spike protein (S protein), such as the S1 domain of the spike protein (S1 protein).

4. The multimeric binding complex according to any one of claims 1 to 3, wherein said loop sequences comprise 2, 3, 4, 5, 6, 7 or 8 amino acids.

5. The multimeric binding complex according to any one of claims 1 to 4, wherein said loop sequences comprise three reactive groups separated by two loop sequences one of which consists of 3 amino acids and the other of which consists of 6 amino acids, such as:
wherein the bicyclic peptide ligand comprises an amino acid sequence which is selected from:
(SEQ ID NO: 1)
CᵢHHACᵢᵢPILTGWCᵢᵢᵢ;
(SEQ ID NO: 6)
CᵢPHACᵢᵢPSLWGWCᵢᵢᵢ;
(SEQ ID NO: 7)
CᵢLHACᵢᵢPRLTHWCᵢᵢᵢ;
(SEQ ID NO: 8)
CᵢLHACᵢᵢQYLWGYCᵢᵢᵢ;
(SEQ ID NO: 9)
CᵢSHACᵢᵢPRLFGWCᵢᵢᵢ;
(SEQ ID NO: 10)
CᵢQHACᵢᵢPYLWDYCᵢᵢᵢ;
(SEQ ID NO: 58)
CᵢPFACᵢᵢHKLYGWCᵢᵢᵢ;
(SEQ ID NO: 59)
CᵢMKACᵢᵢPYLYGWCᵢᵢᵢ;
(SEQ ID NO: 60)
CᵢRHACᵢᵢTHLYGHCᵢᵢᵢ;
(SEQ ID NO: 61)
CᵢPYACᵢᵢTRLYGWCᵢᵢᵢ;
(SEQ ID NO: 62)
CᵢSHACᵢᵢPRLTGWCᵢᵢᵢ;
(SEQ ID NO: 63)
CᵢLHSCᵢᵢPRLSGWCᵢᵢᵢ;
(SEQ ID NO: 64)
CᵢRHSCᵢᵢPILTGWCᵢᵢᵢ;
(SEQ ID NO: 65)
CᵢGHSCᵢᵢPVLWGWCᵢᵢᵢ;
(SEQ ID NO: 66)
CᵢPHSCᵢᵢPKLFGWCᵢᵢᵢ;
and
(SEQ ID NO: 67)
CᵢTHSCᵢᵢPYLFGWCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA and the bicyclic peptide ligand additionally comprises N- and/or C-terminal additions and comprises an amino acid sequence which is selected from:
A-(SEQ ID NO: 1)-A (herein referred to as BCY15230);
A-(SEQ ID NO: 6)-A (herein referred to as BCY15235);
A-(SEQ ID NO: 7)-A (herein referred to as BCY15236);
A-(SEQ ID NO: 8)-A (herein referred to as BCY15237);
A-(SEQ ID NO: 9)-A (herein referred to as BCY15238);
A-(SEQ ID NO: 10)-A (herein referred to as BCY15239);
A-(SEQ ID NO: 58)-A (herein referred to as BCY15364);
A-(SEQ ID NO: 59)-A (herein referred to as BCY15365);
A-(SEQ ID NO: 60)-A (herein referred to as BCY15366);
A-(SEQ ID NO: 61)-A (herein referred to as BCY15367);
A-(SEQ ID NO: 62)-A (herein referred to as BCY15368);
A-(SEQ ID NO: 63)-A (herein referred to as BCY15369);
A-(SEQ ID NO: 64)-A (herein referred to as BCY15370);
A-(SEQ ID NO: 65)-A (herein referred to as BCY15371);
A-(SEQ ID NO: 66)-A (herein referred to as BCY15372); and
A-(SEQ ID NO: 67)-A (herein referred to as BCY15373);
or wherein the molecular scaffold is TATA, the bicyclic peptide additionally comprises N-and/or C-terminal additions and a labelling moiety, such as fluorescein (Fl), and comprises an amino acid sequence which is selected from:
A-(SEQ ID NO: 6)-A-[Sar₆]-[KFl] (herein referred to as BCY15303); and
A-(SEQ ID NO: 63)-A-[Sar₆]-[KFl] (herein referred to as BCY15329).

6. The multimeric binding complex according to any one of claims 1 to 4, wherein said loop sequences comprise three reactive groups separated by two loop sequences one of which consists of 4 amino acids and the other of which consists of 6 amino acids, such as:
wherein the bicyclic peptide ligand comprises an amino acid sequence which is selected from:
(SEQ ID NO: 29)
CᵢLTNDCᵢᵢHSDIRYCᵢᵢᵢ;
and
(SEQ ID NO: 30)
CᵢITNDCᵢᵢHTSLIFCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TBMT and the bicyclic peptide ligand additionally comprises N- and/or C-terminal additions and comprises an amino acid sequence which is selected from:
A-(SEQ ID NO: 29)-A (herein referred to as BCY15335); and
A-(SEQ ID NO: 30)-A (herein referred to as BCY15336);
or wherein the molecular scaffold is TBMT, the bicyclic peptide additionally comprises N-and/or C-terminal additions and a labelling moiety, such as fluorescein (Fl), and comprises an amino acid sequence which is:
A-(SEQ ID NO: 30)-A-[Sar₆]-[KFl] (herein referred to as BCY15314).

7. The multimeric binding complex according to any one of claims 1 to 4, wherein said loop sequences comprise three reactive groups separated by two loop sequences one of which consists of 4 amino acids and the other of which consists of 8 amino acids, such as:
wherein the bicyclic peptide ligand comprises an amino acid sequence which is selected from:
(SEQ ID NO: 3)
CᵢVDANCᵢᵢKIKILQRMCᵢᵢᵢ;
(SEQ ID NO: 4)
CᵢTSSVCᵢᵢKIKELQRKCᵢᵢᵢ;
(SEQ ID NO: 5)
CᵢRSLLCᵢᵢEYLQRTDSCᵢᵢᵢ;
(SEQ ID NO: 14)
CᵢLTKSCᵢᵢKIKMLQRVCᵢᵢᵢ;
(SEQ ID NO: 15)
CᵢMQPSCᵢᵢRVLQLQRVCᵢᵢᵢ;
(SEQ ID NO: 16)
CᵢALPSCᵢᵢRILHLQHRCᵢᵢᵢ;
(SEQ ID NO: 17)
CᵢHDAHCᵢᵢKILELQHRCᵢᵢᵢ;
(SEQ ID NO: 18)
CᵢTSSHCᵢᵢRVLEEQRLCᵢᵢᵢ;
(SEQ ID NO: 19)
CᵢPRDRCᵢᵢPTAWLYGLCᵢᵢᵢ;
(SEQ ID NO: 20)
CᵢAEAGCᵢᵢRVKQLQQICᵢᵢᵢ;
(SEQ ID NO: 21)
CᵢTPSPCᵢᵢRVKELQRACᵢᵢᵢ;
(SEQ ID NO: 26)
CᵢSTANCᵢᵢRILELQQLCᵢᵢᵢ;
(SEQ ID NO: 44)
CᵢVGRLCᵢᵢSTATDIRKCᵢᵢᵢ;
(SEQ ID NO: 48;
herein referred to as BCY16983
when complexed with TATB)
CᵢRQSQCᵢᵢDWWAIRSFCᵢᵢᵢ;
(SEQ ID NO: 49)
CᵢTDATCᵢᵢSIKRLQRLCᵢᵢᵢ;
(SEQ ID NO: 50)
CᵢSPVSCᵢᵢPSGFKFGLCᵢᵢᵢ;
(SEQ ID NO: 68)
CᵢDSPWCᵢᵢRIRSLQRQCᵢᵢᵢ;
(SEQ ID NO: 69)
CᵢSVGACᵢᵢRVKLLQRVCᵢᵢᵢ;
and
(SEQ ID NO: 70)
CᵢMFVPCᵢᵢAVREILGLCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATB and the bicyclic peptide ligand additionally comprises N- and/or C-terminal additions and comprises an amino acid sequence which is selected from:
A-(SEQ ID NO: 3)-A (herein referred to as BCY15334);
A-(SEQ ID NO: 15)-A (herein referred to as BCY15244);
A-(SEQ ID NO: 16)-A (herein referred to as BCY15245);
A-(SEQ ID NO: 17)-A (herein referred to as BCY15246);
A-(SEQ ID NO: 18)-A (herein referred to as BCY15247);
A-(SEQ ID NO: 19)-A (herein referred to as BCY15248);
A-(SEQ ID NO: 20)-A (herein referred to as BCY15249);
A-(SEQ ID NO: 21)-A (herein referred to as BCY15250);
A-(SEQ ID NO: 26)-A (herein referred to as BCY15255);
A-(SEQ ID NO: 48)-A (herein referred to as BCY15354);
A-(SEQ ID NO: 48)-A (herein referred to as BCY16534);
A-(SEQ ID NO: 48)-AK (herein referred to as BCY16896);
A-(SEQ ID NO: 49)-A (herein referred to as BCY15355); and
A-(SEQ ID NO: 50)-A (herein referred to as BCY15356);
or wherein the molecular scaffold is TATB, the bicyclic peptide additionally comprises N-and/or C-terminal additions and a labelling moiety, such as fluorescein (Fl), and comprises an amino acid sequence which is selected from:
A-(SEQ ID NO: 3)-A-[Sar₆]-[KFl] (herein referred to as BCY15301);
A-(SEQ ID NO: 15)-A-[Sar₆]-[KFl] (herein referred to as BCY15307);
A-(SEQ ID NO: 17)-A-[Sar₆]-[KFl] (herein referred to as BCY15308);
A-(SEQ ID NO: 19)-A-[Sar₆]-[KFl] (herein referred to as BCY15309);
A-(SEQ ID NO: 48)-A-[Sar₆]-[KFl] (herein referred to as BCY15324);
A-(SEQ ID NO: 49)-A-[Sar₆]-[KFl] (herein referred to as BCY15325); and
A-(SEQ ID NO: 50)-A-[Sar₆]-[KFl] (herein referred to as BCY15326);
or wherein the molecular scaffold is TATA, the bicyclic peptide ligand additionally comprises N- and/or C-terminal additions and comprises an amino acid sequence which is selected from:
A-(SEQ ID NO: 3)-A (herein referred to as BCY15232);
A-(SEQ ID NO: 4)-A (herein referred to as BCY15233);
A-(SEQ ID NO: 5)-A (herein referred to as BCY15234);
A-(SEQ ID NO: 14)-A (herein referred to as BCY15243);
A-(SEQ ID NO: 44)-A (herein referred to as BCY15350);
A-(SEQ ID NO: 68)-A (herein referred to as BCY15374);
A-(SEQ ID NO: 69)-A (herein referred to as BCY15375); and
A-(SEQ ID NO: 70)-A (herein referred to as BCY15376);
or wherein the molecular scaffold is TATA, the bicyclic peptide additionally comprises N-and/or C-terminal additions and a labelling moiety, such as fluorescein (Fl), and comprises an amino acid sequence which is selected from:
A-(SEQ ID NO: 3)-A-[Sar₆]-[KFl] (herein referred to as BCY15300);
A-(SEQ ID NO: 5)-A-[Sar₆]-[KFl] (herein referred to as BCY15302); and
A-(SEQ ID NO: 70)-A-[Sar₆]-[KFl] (herein referred to as BCY15330);
or wherein the molecular scaffold is TBMT and the bicyclic peptide ligand additionally comprises N- and/or C-terminal additions and comprises an amino acid sequence which is:
A-(SEQ ID NO: 48)-AK (herein referred to as BCY16986).

8. The multimeric binding complex according to any one of claims 1 to 4, wherein said loop sequences comprise three reactive groups separated by two loop sequences one of which consists of 6 amino acids and the other of which consists of 3 amino acids, such as:
wherein the bicyclic peptide ligand comprises an amino acid sequence which is selected from:
(SEQ ID NO: 71)
CᵢTLMDPWCᵢᵢLLKCᵢᵢᵢ;
(SEQ ID NO: 72)
CᵢKIHDWTCᵢᵢLLRCᵢᵢᵢ;
and
(SEQ ID NO: 79)
CᵢIPLDWTCᵢᵢMIACᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA and the bicyclic peptide ligand additionally comprises N- and/or C-terminal additions and comprises an amino acid sequence which is selected from:
A-(SEQ ID NO: 71)-A (herein referred to as BCY15377); and
A-(SEQ ID NO: 72)-A (herein referred to as BCY15378);
or wherein the molecular scaffold is TATA, the bicyclic peptide additionally comprises N-and/or C-terminal additions and a labelling moiety, such as fluorescein (Fl), and comprises an amino acid sequence which is:
A-(SEQ ID NO: 71)-A-[Sar₆]-[KFl] (herein referred to as BCY15331);
or wherein the molecular scaffold is TATB and the bicyclic peptide ligand additionally comprises N- and/or C-terminal additions and comprises an amino acid sequence which is selected from:
Ac-(SEQ ID NO: 79) (herein referred to as BCY16991);
A-(SEQ ID NO: 79)-A (herein referred to as BCY15446);
A-(SEQ ID NO: 79)-AK (herein referred to as BCY16994); and
Ac-(SEQ ID NO: 79)-K (herein referred to as BCY18654).

9. The multimeric binding complex according to any one of claims 1 to 4, wherein said loop sequences comprise three reactive groups separated by two loop sequences one of which consists of 6 amino acids and the other of which consists of 4 amino acids, such as:
wherein the bicyclic peptide ligand comprises an amino acid sequence which is:
(SEQ ID NO: 11)
CᵢEYQGPHCᵢᵢYRLYCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA, the bicyclic peptide ligand additionally comprises N-and/or C-terminal additions and comprises an amino acid sequence which is:
A-(SEQ ID NO: 11)-A (herein referred to as BCY15240);
or wherein the molecular scaffold is TATA, the bicyclic peptide additionally comprises N-and/or C-terminal additions and a labelling moiety, such as fluorescein (Fl), and comprises an amino acid sequence which is:
A-(SEQ ID NO: 11)-A-[Sar₆]-[KFl] (herein referred to as BCY15304).

10. The multimeric binding complex according to any one of claims 1 to 4, wherein said loop sequences comprise three reactive groups separated by two loop sequences one of which consists of 7 amino acids and the other of which consists of 2 amino acids, such as:
wherein the bicyclic peptide ligand comprises an amino acid sequence which is selected from:
(SEQ ID NO: 2)
CᵢEDHDWVYCᵢᵢSTCᵢᵢᵢ;
(SEQ ID NO: 23)
CᵢAPWNYFRCᵢᵢDLCᵢᵢᵢ;
(SEQ ID NO: 25)
CᵢLTPEDIWCᵢᵢMLCᵢᵢᵢ;
(SEQ ID NO: 28)
CᵢENPVDIWCᵢᵢVLCᵢᵢᵢ;
(SEQ ID NO: 46)
CᵢVFTTVWDCᵢᵢLACᵢᵢᵢ;
(SEQ ID NO: 51)
CᵢYDPIDVWCᵢᵢMMCᵢᵢᵢ;
(SEQ ID NO: 52)
CᵢASYDDFWCᵢᵢVLCᵢᵢᵢ;
(SEQ ID NO: 53)
CᵢDLTQHWTCᵢᵢILCᵢᵢᵢ;
(SEQ ID NO: 54)
CᵢSEISDVWCᵢᵢMLCᵢᵢᵢ;
(SEQ ID NO: 55)
CᵢPTPVDIWCᵢᵢMLCᵢᵢᵢ;
(SEQ ID NO: 73)
CᵢEQNGWIYCᵢᵢSTCᵢᵢᵢ;
(SEQ ID NO: 74)
CᵢTDRSWIFCᵢᵢSTCᵢᵢᵢ;
and
(SEQ ID NO: 75)
CᵢPNISWIYCᵢᵢSTCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA, the bicyclic peptide ligand additionally comprises N-and/or C-terminal additions and comprises an amino acid sequence which is selected from:
A-(SEQ ID NO: 2)-A (herein referred to as BCY15231);
Ac-(SEQ ID NO: 2) (herein referred to as BCY16987);
A-(SEQ ID NO: 2)-AK (herein referred to as BCY16990);
A-(SEQ ID NO: 46)-A (herein referred to as BCY15352);
A-(SEQ ID NO: 73)-A (herein referred to as BCY15379);
A-(SEQ ID NO: 74)-A (herein referred to as BCY15380); and
A-(SEQ ID NO: 75)-A (herein referred to as BCY15381);
or wherein the molecular scaffold is TATA, the bicyclic peptide additionally comprises N-and/or C-terminal additions and a labelling moiety, such as fluorescein (Fl), and comprises an amino acid sequence which is selected from:
A-(SEQ ID NO: 2)-A-[Sar₆]-[KFl] (herein referred to as BCY15299); and
A-(SEQ ID NO: 74)-A-[Sar₆]-[KFl] (herein referred to as BCY15332);
or wherein the molecular scaffold is TATB, the bicyclic peptide ligand additionally comprises N- and/or C-terminal additions and comprises an amino acid sequence which is selected from:
A-(SEQ ID NO: 23)-A (herein referred to as BCY15252);
A-(SEQ ID NO: 25)-A (herein referred to as BCY15254);
A-(SEQ ID NO: 28)-A (herein referred to as BCY15257);
A-(SEQ ID NO: 51)-A (herein referred to as BCY15357);
A-(SEQ ID NO: 52)-A (herein referred to as BCY15358);
A-(SEQ ID NO: 53)-A (herein referred to as BCY15359);
A-(SEQ ID NO: 54)-A (herein referred to as BCY15360); and
A-(SEQ ID NO: 55)-A (herein referred to as BCY15361);
or wherein the molecular scaffold is TATB, the bicyclic peptide additionally comprises N-and/or C-terminal additions and a labelling moiety, such as fluorescein (Fl), and comprises an amino acid sequence which is selected from:
A-(SEQ ID NO: 23)-A-[Sar₆]-[KFl] (herein referred to as BCY15311);
A-(SEQ ID NO: 25)-A-[Sar₆]-[KFl] (herein referred to as BCY15312); and
A-(SEQ ID NO: 53)-A-[Sar₆]-[KFl] (herein referred to as BCY15327).

11. The multimeric binding complex according to any one of claims 1 to 4, wherein said loop sequences comprise three reactive groups separated by two loop sequences one of which consists of 7 amino acids and the other of which consists of 3 amino acids, such as:
wherein the bicyclic peptide ligand comprises an amino acid sequence which is selected from:
(SEQ ID NO: 22; herein referred to as BCY16534
when complexed with TATB)
CᵢASPDNPVCᵢᵢRFYCᵢᵢᵢ;
(SEQ ID NO: 24 herein referred to as BCY16540
when complexed with TATB)
CᵢYNHANPVCᵢᵢRYYCᵢᵢᵢ;
(SEQ ID NO: 27)
CᵢDLFLHELCᵢᵢDMPCᵢᵢᵢ;
(SEQ ID NO: 31)
CᵢNKQNWRYCᵢᵢYLTCᵢᵢᵢ;
(SEQ ID NO: 56)
CᵢHPWSALFCᵢᵢNYPCᵢᵢᵢ;
(SEQ ID NO: 57)
CᵢYAPDNPVCᵢᵢRMYCᵢᵢᵢ;
(SEQ ID NO: 76)
CᵢGILADPFCᵢᵢLISCᵢᵢᵢ;
(SEQ ID NO: 89)
CᵢYNHANPVCᵢᵢ[Agb]YYCᵢᵢᵢ;
(SEQ ID NO: 90)
CᵢASPDNPVCᵢᵢ[Agb]FYCᵢᵢᵢ;
(SEQ ID NO: 91)
CᵢASPDNPVCᵢᵢ[Arg(Me)]FYCᵢᵢᵢ;
(SEQ ID NO: 92)
CᵢCASPDNPVCᵢᵢ[HArg]FYCᵢᵢᵢ;
(SEQ ID NO: 93)
CᵢANPDNPVCᵢᵢRFYCᵢᵢᵢ;
(SEQ ID NO: 94)
CᵢRNPDNPVCᵢᵢRFYCᵢᵢᵢ;
(SEQ ID NO: 95)
CᵢHNPSNPVCᵢᵢRFYCᵢᵢᵢ;
(SEQ ID NO: 96)
CᵢVNKHNPVCᵢᵢRFYCᵢᵢᵢ;
(SEQ ID NO: 97)
CᵢVNAENPVCᵢᵢRFYCᵢᵢᵢ;
(SEQ ID NO: 98)
CᵢQNPGNPVCᵢᵢRFYCᵢᵢᵢ;
(SEQ ID NO: 99)
CᵢMNPDNPVCᵢᵢRFYCᵢᵢᵢ;
(SEQ ID NO: 100)
CᵢYNQENPVCᵢᵢRFYCᵢᵢᵢ;
(SEQ ID NO: 101)
CᵢNNPANPVCᵢᵢRFYCᵢᵢᵢ;
(SEQ ID NO: 102)
CᵢFNIDNPVCᵢᵢRFYCᵢᵢᵢ;
(SEQ ID NO: 103)
CᵢSNPENPVCᵢᵢRFYCᵢᵢᵢ;
(SEQ ID NO: 104)
CᵢMNEDNPVCᵢᵢRFYCᵢᵢᵢ;
(SEQ ID NO: 105)
CᵢMNEANPVCᵢᵢRFYCᵢᵢᵢ;
(SEQ ID NO: 106)
CᵢHNLDNPVCᵢᵢRFYCᵢᵢᵢ;
(SEQ ID NO: 107)
CᵢANHDNPVCᵢᵢRFYCᵢᵢᵢ;
(SEQ ID NO: 108)
CᵢKNYDNPVCᵢᵢRFYCᵢᵢᵢ;
(SEQ ID NO: 109)
CᵢENMDNPVCᵢᵢRFYCᵢᵢᵢ;
(SEQ ID NO: 110)
CᵢMNTDNPVCᵢᵢRFYCᵢᵢᵢ;
(SEQ ID NO: 111)
CᵢLNVDNPVCᵢᵢRFYCᵢᵢᵢ;
(SEQ ID NO: 112)
CᵢLNPDNPVCᵢᵢRFYCᵢᵢᵢ;
(SEQ ID NO: 113)
CᵢYNHANPVCᵢᵢ[HArg]YYCᵢᵢᵢ;
and
(SEQ ID NO: 114)
CᵢYNHANPVCᵢᵢ[Arg(Me)]YYCᵢᵢᵢ;
wherein Cᵢ, Cᵢᵢ and Cᵢᵢᵢ represent first, second and third cysteine residues, respectively, Agb represents 2-amino-4-guanidinobutyric acid, Arg(Me) represents d-N methyl arginine, HArg represents homoarginine, or a pharmaceutically acceptable salt thereof, in particular:
wherein the molecular scaffold is TATB, the bicyclic peptide ligand additionally comprises N-and/or C-terminal additions and comprises an amino acid sequence which is selected from:
A-(SEQ ID NO: 22)-A (herein referred to as BCY15251);
Ac-A-(SEQ ID NO: 22)-A (herein referred to as BCY16538);
Ac-(SEQ ID NO: 22) (herein referred to as BCY15576);
Ac-A-(SEQ ID NO: 22)-AK (herein referred to as BCY16982);
Ac-A-(SEQ ID NO: 24)-A (herein referred to as BCY16545);
Ac-(SEQ ID NO: 24) (herein referred to as BCY16544);
A-(SEQ ID NO: 24)-A (herein referred to as BCY15522);
A-(SEQ ID NO: 27)-A (herein referred to as BCY15256);
A-(SEQ ID NO: 56)-A (herein referred to as BCY15362);
A-(SEQ ID NO: 57)-A (herein referred to as BCY15363);
A-(SEQ ID NO: 89)-A (herein referred to as BCY16541);
A-(SEQ ID NO: 90)-A (herein referred to as BCY16535);
A-(SEQ ID NO: 91)-A (herein referred to as BCY16536);
A-(SEQ ID NO: 92)-A (herein referred to as BCY16537);
Ac-(SEQ ID NO: 93) (herein referred to as BCY16903);
Ac-(SEQ ID NO: 93)-K (herein referred to as BCY18579);
Ac-(SEQ ID NO: 94) (herein referred to as BCY16905);
Ac-(SEQ ID NO: 95) (herein referred to as BCY16906);
Ac-(SEQ ID NO: 96) (herein referred to as BCY16911);
Ac-(SEQ ID NO: 97) (herein referred to as BCY16913);
Ac-(SEQ ID NO: 98) (herein referred to as BCY16915);
Ac-(SEQ ID NO: 99) (herein referred to as BCY16917);
Ac-(SEQ ID NO: 100) (herein referred to as BCY16918);
Ac-(SEQ ID NO: 101) (herein referred to as BCY16921);
Ac-(SEQ ID NO: 102) (herein referred to as BCY16912);
Ac-(SEQ ID NO: 103) (herein referred to as BCY16914);
Ac-(SEQ ID NO: 104) (herein referred to as BCY16916);
Ac-(SEQ ID NO: 105) (herein referred to as BCY16919);
Ac-(SEQ ID NO: 106) (herein referred to as BCY16920);
Ac-(SEQ ID NO: 107) (herein referred to as BCY16902);
Ac-(SEQ ID NO: 108) (herein referred to as BCY16904);
Ac-(SEQ ID NO: 109) (herein referred to as BCY16907);
Ac-(SEQ ID NO: 110) (herein referred to as BCY16908);
Ac-(SEQ ID NO: 111) (herein referred to as BCY16909);
Ac-(SEQ ID NO: 112) (herein referred to as BCY16910);
A-(SEQ ID NO: 113)-A (herein referred to as BCY16543); and
A-(SEQ ID NO: 114)-A (herein referred to as BCY16542).
or wherein the molecular scaffold is TATB, the bicyclic peptide additionally comprises N-and/or C-terminal additions and a labelling moiety, such as fluorescein (Fl), and comprises an amino acid sequence which is selected from:
A-(SEQ ID NO: 22)-A-[Sar₆]-[KFl] (herein referred to as BCY15310);
A-(SEQ ID NO: 27)-A-[Sar₆]-[KFl] (herein referred to as BCY15313); and
A-(SEQ ID NO: 56)-A-[Sar₆]-[KFl] (herein referred to as BCY15328);
or wherein the molecular scaffold is TBMT, the bicyclic peptide ligand additionally comprises N- and/or C-terminal additions and comprises an amino acid sequence which is:
A-(SEQ ID NO: 31)-A (herein referred to as BCY15315);
or wherein the molecular scaffold is TBMT, the bicyclic peptide additionally comprises N-and/or C-terminal additions and a labelling moiety, such as fluorescein (Fl), and comprises an amino acid sequence which is:
A-(SEQ ID NO: 31)-A-[Sar₆]-[KFl] (herein referred to as BCY15313);
or wherein the molecular scaffold is TATA, the bicyclic peptide ligand additionally comprises N- and/or C-terminal additions and comprises an amino acid sequence which is:
A-(SEQ ID NO: 76)-A (herein referred to as BCY15382);
or wherein the molecular scaffold is TATA, the bicyclic peptide additionally comprises N-and/or C-terminal additions and a labelling moiety, such as fluorescein (Fl), and comprises an amino acid sequence which is:
A-(SEQ ID NO: 76)-A-[Sar₆]-[KFl] (herein referred to as BCY15333).

12. The multimeric binding complex according to any one of claims 1 to 4, wherein said loop sequences comprise three reactive groups separated by two loop sequences one of which consists of 7 amino acids and the other of which consists of 5 amino acids, such as:
wherein the bicyclic peptide ligand comprises an amino acid sequence which is selected from:
(SEQ ID NO: 32)
CᵢTTSEKVKCᵢᵢLQRHPCᵢᵢᵢ;
(SEQ ID NO: 33)
CᵢQPDMRIKCᵢᵢLQRVACᵢᵢᵢ;
(SEQ ID NO: 34)
CᵢSSNNRIKCᵢᵢLQRVTCᵢᵢᵢ;
and
(SEQ ID NO: 35)
CᵢKEKTTIGCᵢᵢLMAGICᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TBMT, the bicyclic peptide ligand additionally comprises N-and/or C-terminal additions and comprises an amino acid sequence which is selected from:
A-(SEQ ID NO: 32)-A (herein referred to as BCY15338);
A-(SEQ ID NO: 33)-A (herein referred to as BCY15339);
A-(SEQ ID NO: 34)-A (herein referred to as BCY15340); and
A-(SEQ ID NO: 35)-A (herein referred to as BCY15341);
or wherein the molecular scaffold is TBMT, the bicyclic peptide additionally comprises N-and/or C-terminal additions and a labelling moiety, such as fluorescein (Fl), and comprises an amino acid sequence which is selected from:
A-(SEQ ID NO: 32)-A-[Sar₆]-[KFl] (herein referred to as BCY15316); and
A-(SEQ ID NO: 33)-A-[Sar₆]-[KFl] (herein referred to as BCY15317).

13. The multimeric binding complex according to any one of claims 1 to 4, wherein said loop sequences comprise three reactive groups separated by two loop sequences one of which consists of 8 amino acids and the other of which consists of 2 amino acids, such as:
wherein the bicyclic peptide ligand comprises an amino acid sequence which is selected from:
(SEQ ID NO: 12)
CᵢGRDSSWIYCᵢᵢSTCᵢᵢᵢ;
(SEQ ID NO: 13)
CᵢRGTPAWKACᵢᵢAICᵢᵢᵢ;
(SEQ ID NO: 36)
CᵢPFPSGFGTCᵢᵢTFCᵢᵢᵢ;
(SEQ ID NO: 37; herein referred to as BCY16312
when complexed with TBMT)
CᵢPYVAGRGTCᵢᵢLLCᵢᵢᵢ;
(SEQ ID NO: 38)
CᵢPYPRGTGSCᵢᵢTFCᵢᵢᵢ;
(SEQ ID NO: 39)
CᵢLYPPGKGTCᵢᵢLLCᵢᵢᵢ;
(SEQ ID NO: 40)
CᵢPSPAGRGTCᵢᵢLLCᵢᵢᵢ;
(SEQ ID NO: 41)
CᵢPATIGRGPCᵢᵢTFCᵢᵢᵢ;
(SEQ ID NO: 77)
CᵢPEANSWVYCᵢᵢSTCᵢᵢᵢ;
(SEQ ID NO: 78)
CᵢAPTSGWIYCᵢᵢSTCᵢᵢᵢ;
(SEQ ID NO: 80)
CᵢPYVAG[Agb]GTCᵢᵢLLCᵢᵢᵢ;
(SEQ ID NO: 81)
CᵢPYVAG[Arg(Me)]GTCᵢᵢLLCᵢᵢᵢ;
(SEQ ID NO: 82)
CᵢPYVAGRGTCᵢᵢL[Cba]Cᵢᵢᵢ;
(SEQ ID NO: 83)
CᵢPYVAGRGTCᵢᵢ[Cba]LCᵢᵢᵢ;
(SEQ ID NO: 84)
CᵢPYVAGR[dA]TCᵢᵢLLCᵢᵢᵢ;
(SEQ ID NO: 85)
CᵢPYVAG[HArg]GTCᵢᵢLLCᵢᵢᵢ;
(SEQ ID NO: 86)
CᵢPYVAGRGTCᵢᵢL[tBuAla]Cᵢᵢᵢ;
(SEQ ID NO: 87)
CᵢPYVAGRGTCᵢᵢ[tBuAla]LCᵢᵢᵢ;
(SEQ ID NO: 88)
CᵢPYVAG[Agb][dA]TCᵢᵢL[tBuAla]Cᵢᵢᵢ;
and
(SEQ ID NO: 115)
CᵢPYVAG[HArg][dA]TCᵢᵢL[tBuAla]Cᵢᵢᵢ;
wherein Cᵢ, Cᵢᵢ and Cᵢᵢᵢ represent first, second and third cysteine residues, respectively, Agb represents 2-amino-4-guanidinobutyric acid, Arg(Me) represents d-N methyl arginine, Cba represents β-cyclobutylalanine, HArg represents homoarginine, tBuAla represents t-butyl-alanine, or a pharmaceutically acceptable salt thereof, in particular:
wherein the molecular scaffold is TATA, the bicyclic peptide ligand additionally comprises N- and/or C-terminal additions and comprises an amino acid sequence which is selected from:
A-(SEQ ID NO: 12)-A (herein referred to as BCY15241);
A-(SEQ ID NO: 13)-A (herein referred to as BCY15242);
A-(SEQ ID NO: 77)-A (herein referred to as BCY15383); and
A-(SEQ ID NO: 78)-A (herein referred to as BCY15384);
or wherein the molecular scaffold is TATA, the bicyclic peptide additionally comprises N- and/or C-terminal additions and a labelling moiety, such as fluorescein (Fl), and comprises an amino acid sequence which is selected from:
A-(SEQ ID NO: 12)-A-[Sar₆]-[KFl] (herein referred to as BCY15305); and
A-(SEQ ID NO: 13)-A-[Sar₆]-[KFl] (herein referred to as BCY15306);
or wherein the molecular scaffold is TBMT, the bicyclic peptide ligand additionally comprises N- and/or C-terminal additions and comprises an amino acid sequence which is selected from:
A-(SEQ ID NO: 36)-A (herein referred to as BCY15342);
Ac-A-(SEQ ID NO: 37)-A (herein referred to as BCY16322);
Ac-(SEQ ID NO: 37) (herein referred to as BCY16323);
A-(SEQ ID NO: 37)-A (herein referred to as BCY15343);
A-(SEQ ID NO: 38)-A (herein referred to as BCY15344);
A-(SEQ ID NO: 39)-A (herein referred to as BCY15345);
A-(SEQ ID NO: 40)-A (herein referred to as BCY15346);
A-(SEQ ID NO: 41)-A (herein referred to as BCY15347);
A-(SEQ ID NO: 80)-A (herein referred to as BCY16313);
A-(SEQ ID NO: 81)-A (herein referred to as BCY16314);
A-(SEQ ID NO: 82)-A (herein referred to as BCY16315);
A-(SEQ ID NO: 83)-A (herein referred to as BCY16316);
A-(SEQ ID NO: 84)-A (herein referred to as BCY16318);
A-(SEQ ID NO: 85)-A (herein referred to as BCY16319);
A-(SEQ ID NO: 86)-A (herein referred to as BCY16320);
A-(SEQ ID NO: 87)-A (herein referred to as BCY16321);
Ac-(SEQ ID NO: 88) (herein referred to as BCY16591);
Ac-(SEQ ID NO: 88)-[K(PYA)] (herein referred to as BCY16592);
Ac-(SEQ ID NO: 88)-K (herein referred to as BCY17018); and
Ac-(SEQ ID NO: 115) (herein referred to as BCY18024), wherein PYA represents pentynoic acid;
or wherein the molecular scaffold is TBMT, the bicyclic peptide additionally comprises N-and/or C-terminal additions and a labelling moiety, such as fluorescein (Fl), and comprises an amino acid sequence which is selected from:
A-(SEQ ID NO: 37)-A-[Sar 6 ]-[KFl] (herein referred to as BCY15318); and
A-(SEQ ID NO: 38)-A-[Sar 6 ]-[KFl] (herein referred to as BCY15319).

14. The multimeric binding complex according to any one of claims 1 to 4, wherein said loop sequences comprise three reactive groups separated by two loop sequences one of which consists of 8 amino acids and the other of which consists of 3 amino acids, such as:
wherein the bicyclic peptide ligand comprises an amino acid sequence which is selected from:
(SEQ ID NO: 45)
CᵢSNTWHWTDCᵢᵢLAECᵢᵢᵢ;
and
(SEQ ID NO: 47)
CᵢNLWNGDPWCᵢᵢLLRCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TATA, the bicyclic peptide ligand additionally comprises N-and/or C-terminal additions and comprises an amino acid sequence which is selected from:
A-(SEQ ID NO: 45)-A (herein referred to as BCY15351); and
A-(SEQ ID NO: 47)-A (herein referred to as BCY15353);
or wherein the molecular scaffold is TATA, the bicyclic peptide additionally comprises N-and/or C-terminal additions and a labelling moiety, such as fluorescein (Fl), and comprises an amino acid sequence which is selected from:
A-(SEQ ID NO: 45)-A-[Sar₆]-[KFl] (herein referred to as BCY15322); and
A-(SEQ ID NO: 47)-A-[Sar₆]-[KFl] (herein referred to as BCY15323).

15. The multimeric binding complex according to any one of claims 1 to 4, wherein said loop sequences comprise three reactive groups separated by two loop sequences one of which consists of 8 amino acids and the other of which consists of 4 amino acids, such as:
wherein the bicyclic peptide ligand comprises an amino acid sequence which is selected from:
(SEQ ID NO: 42)
CᵢHQLMDIWDCᵢᵢLRPDCᵢᵢᵢ;
and
(SEQ ID NO: 43)
CᵢLTAREKIQCᵢᵢLQRRCᵢᵢᵢ;
wherein Cᵢ, Cᵢᵢ and Cᵢᵢᵢ represent first, second and third cysteine residues, respectively, or a pharmaceutically acceptable salt thereof, in particular:
wherein the molecular scaffold is TBMT, the bicyclic peptide ligand additionally comprises N-and/or C-terminal additions and comprises an amino acid sequence which is selected from:
A-(SEQ ID NO: 42)-A (herein referred to as BCY15348); and
A-(SEQ ID NO: 43)-A (herein referred to as BCY15349);
or wherein the molecular scaffold is TBMT, the bicyclic peptide additionally comprises N-and/or C-terminal additions and a labelling moiety, such as fluorescein (Fl), and comprises an amino acid sequence which is selected from:
A-(SEQ ID NO: 42)-A-[Sar₆]-[KFl] (herein referred to as BCY15320); and
A-(SEQ ID NO: 43)-A-[Sar₆]-[KFl] (herein referred to as BCY15321).

16. The multimeric binding complex according to any one of claims 1 to 15, which is selected from those listed in Table A, such as BCY18656.

17. The multimeric binding complex according to any one of claims 1 to 16, wherein the pharmaceutically acceptable salt is selected from the free acid or the sodium, potassium, calcium and ammonium salt.

18. A pharmaceutical composition which comprises the multimeric binding complex of any one of claims 1 to 17, in combination with one or more pharmaceutically acceptable excipients.

19. The pharmaceutical composition according to claim 18, which additionally comprises one or more therapeutic agents.

20. The multimeric binding complex according to any of claims 1 to 17, or the pharmaceutical composition as defined in claim 18 or claim 19, for use in suppressing or treating a disease or disorder mediated by infection of SARS-CoV-2 or for providing prophylaxis to a subject at risk of infection of SARS-CoV-2, such as COVID-19.


SEQUENCE LISTING (118 sequences)

SEQ ID NO: 1 - protein, 12 aa, Artificial
  Synthetic Peptide
CHHACPILTGWC

SEQ ID NO: 2 - protein, 12 aa, Artificial
  Synthetic Peptide
CEDHDWVYCSTC

SEQ ID NO: 3 - protein, 15 aa, Artificial
  Synthetic Peptide
CVDANCKIKILQRMC

SEQ ID NO: 4 - protein, 15 aa, Artificial
  Synthetic Peptide
CTSSVCKIKELQRKC

SEQ ID NO: 5 - protein, 15 aa, Artificial
  Synthetic Peptide
CRSLLCEYLQRTDSC

SEQ ID NO: 6 - protein, 12 aa, Artificial
  Synthetic Peptide
CPHACPSLWGWC

SEQ ID NO: 7 - protein, 12 aa, Artificial
  Synthetic Peptide
CLHACPRLTHWC

SEQ ID NO: 8 - protein, 12 aa, Artificial
  Synthetic Peptide
CLHACQYLWGYC

SEQ ID NO: 9 - protein, 12 aa, Artificial
  Synthetic Peptide
CSHACPRLFGWC

SEQ ID NO: 10 - protein, 12 aa, Artificial
  Synthetic Peptide
CQHACPYLWDYC

SEQ ID NO: 11 - protein, 13 aa, Artificial
  Synthetic Peptide
CEYQGPHCYRLYC

SEQ ID NO: 12 - protein, 13 aa, Artificial
  Synthetic Peptide
CGRDSSWIYCSTC

SEQ ID NO: 13 - protein, 13 aa, Artificial
  Synthetic Peptide
CRGTPAWKACAIC

SEQ ID NO: 14 - protein, 15 aa, Artificial
  Synthetic Peptide
CLTKSCKIKMLQRVC

SEQ ID NO: 15 - protein, 15 aa, Artificial
  Synthetic Peptide
CMQPSCRVLQLQRVC

SEQ ID NO: 16 - protein, 15 aa, Artificial
  Synthetic Peptide
CALPSCRILHLQHRC

SEQ ID NO: 17 - protein, 15 aa, Artificial
  Synthetic Peptide
CHDAHCKILELQHRC

SEQ ID NO: 18 - protein, 15 aa, Artificial
  Synthetic Peptide
CTSSHCRVLEEQRLC

SEQ ID NO: 19 - protein, 15 aa, Artificial
  Synthetic Peptide
CPRDRCPTAWLYGLC

SEQ ID NO: 20 - protein, 15 aa, Artificial
  Synthetic Peptide
CAEAGCRVKQLQQIC

SEQ ID NO: 21 - protein, 15 aa, Artificial
  Synthetic Peptide
CTPSPCRVKELQRAC

SEQ ID NO: 22 - protein, 13 aa, Artificial
  Synthetic Peptide
CASPDNPVCRFYC

SEQ ID NO: 23 - protein, 12 aa, Artificial
  Synthetic Peptide
CAPWNYFRCDLC

SEQ ID NO: 24 - protein, 13 aa, Artificial
  Synthetic Peptide
CYNHANPVCRYYC

SEQ ID NO: 25 - protein, 12 aa, Artificial
  Synthetic Peptide
CLTPEDIWCMLC

SEQ ID NO: 26 - protein, 15 aa, Artificial
  Synthetic Peptide
CSTANCRILELQQLC

SEQ ID NO: 27 - protein, 13 aa, Artificial
  Synthetic Peptide
CDLFLHELCDMPC

SEQ ID NO: 28 - protein, 12 aa, Artificial
  Synthetic Peptide
CENPVDIWCVLC

SEQ ID NO: 29 - protein, 13 aa, Artificial
  Synthetic Peptide
CLTNDCHSDIRYC

SEQ ID NO: 30 - protein, 13 aa, Artificial
  Synthetic Peptide
CITNDCHTSLIFC

SEQ ID NO: 31 - protein, 13 aa, Artificial
  Synthetic Peptide
CNKQNWRYCYLTC

SEQ ID NO: 32 - protein, 15 aa, Artificial
  Synthetic Peptide
CTTSEKVKCLQRHPC

SEQ ID NO: 33 - protein, 15 aa, Artificial
  Synthetic Peptide
CQPDMRIKCLQRVAC

SEQ ID NO: 34 - protein, 15 aa, Artificial
  Synthetic Peptide
CSSNNRIKCLQRVTC

SEQ ID NO: 35 - protein, 15 aa, Artificial
  Synthetic Peptide
CKEKTTIGCLMAGIC

SEQ ID NO: 36 - protein, 13 aa, Artificial
  Synthetic Peptide
CPFPSGFGTCTFC

SEQ ID NO: 37 - protein, 13 aa, Artificial
  Synthetic Peptide
CPYVAGRGTCLLC

SEQ ID NO: 38 - protein, 13 aa, Artificial
  Synthetic Peptide
CPYPRGTGSCTFC

SEQ ID NO: 39 - protein, 13 aa, Artificial
  Synthetic Peptide
CLYPPGKGTCLLC

SEQ ID NO: 40 - protein, 13 aa, Artificial
  Synthetic Peptide
CPSPAGRGTCLLC

SEQ ID NO: 41 - protein, 13 aa, Artificial
  Synthetic Peptide
CPATIGRGPCTFC

SEQ ID NO: 42 - protein, 15 aa, Artificial
  Synthetic Peptide
CHQLMDIWDCLRPDC

SEQ ID NO: 43 - protein, 15 aa, Artificial
  Synthetic Peptide
CLTAREKIQCLQRRC

SEQ ID NO: 44 - protein, 15 aa, Artificial
  Synthetic Peptide
CVGRLCSTATDIRKC

SEQ ID NO: 45 - protein, 14 aa, Artificial
  Synthetic Peptide
CSNTWHWTDCLAEC

SEQ ID NO: 46 - protein, 12 aa, Artificial
  Synthetic Peptide
CVFTTVWDCLAC

SEQ ID NO: 47 - protein, 14 aa, Artificial
  Synthetic Peptide
CNLWNGDPWCLLRC

SEQ ID NO: 48 - protein, 15 aa, Artificial
  Synthetic Peptide
CRQSQCDWWAIRSFC

SEQ ID NO: 49 - protein, 15 aa, Artificial
  Synthetic Peptide
CTDATCSIKRLQRLC

SEQ ID NO: 50 - protein, 15 aa, Artificial
  Synthetic Peptide
CSPVSCPSGFKFGLC

SEQ ID NO: 51 - protein, 12 aa, Artificial
  Synthetic Peptide
CYDPIDVWCMMC

SEQ ID NO: 52 - protein, 12 aa, Artificial
  Synthetic Peptide
CASYDDFWCVLC

SEQ ID NO: 53 - protein, 12 aa, Artificial
  Synthetic Peptide
CDLTQHWTCILC

SEQ ID NO: 54 - protein, 12 aa, Artificial
  Synthetic Peptide
CSEISDVWCMLC

SEQ ID NO: 55 - protein, 12 aa, Artificial
  Synthetic Peptide
CPTPVDIWCMLC

SEQ ID NO: 56 - protein, 13 aa, Artificial
  Synthetic Peptide
CHPWSALFCNYPC

SEQ ID NO: 57 - protein, 13 aa, Artificial
  Synthetic Peptide
CYAPDNPVCRMYC

SEQ ID NO: 58 - protein, 12 aa, Artificial
  Synthetic Peptide
CPFACHKLYGWC

SEQ ID NO: 59 - protein, 12 aa, Artificial
  Synthetic Peptide
CMKACPYLYGWC

SEQ ID NO: 60 - protein, 12 aa, Artificial
  Synthetic Peptide
CRHACTHLYGHC

SEQ ID NO: 61 - protein, 12 aa, Artificial
  Synthetic Peptide
CPYACTRLYGWC

SEQ ID NO: 62 - protein, 12 aa, Artificial
  Synthetic Peptide
CSHACPRLTGWC

SEQ ID NO: 63 - protein, 12 aa, Artificial
  Synthetic Peptide
CLHSCPRLSGWC

SEQ ID NO: 64 - protein, 12 aa, Artificial
  Synthetic Peptide
CRHSCPILTGWC

SEQ ID NO: 65 - protein, 12 aa, Artificial
  Synthetic Peptide
CGHSCPVLWGWC

SEQ ID NO: 66 - protein, 12 aa, Artificial
  Synthetic Peptide
CPHSCPKLFGWC

SEQ ID NO: 67 - protein, 12 aa, Artificial
  Synthetic Peptide
CTHSCPYLFGWC

SEQ ID NO: 68 - protein, 15 aa, Artificial
  Synthetic Peptide
CDSPWCRIRSLQRQC

SEQ ID NO: 69 - protein, 15 aa, Artificial
  Synthetic Peptide
CSVGACRVKLLQRVC

SEQ ID NO: 70 - protein, 15 aa, Artificial
  Synthetic Peptide
CMFVPCAVREILGLC

SEQ ID NO: 71 - protein, 12 aa, Artificial
  Synthetic Peptide
CTLMDPWCLLKC

SEQ ID NO: 72 - protein, 12 aa, Artificial
  Synthetic Peptide
CKIHDWTCLLRC

SEQ ID NO: 73 - protein, 12 aa, Artificial
  Synthetic Peptide
CEQNGWIYCSTC

SEQ ID NO: 74 - protein, 12 aa, Artificial
  Synthetic Peptide
CTDRSWIFCSTC

SEQ ID NO: 75 - protein, 12 aa, Artificial
  Synthetic Peptide
CPNISWIYCSTC

SEQ ID NO: 76 - protein, 13 aa, Artificial
  Synthetic Peptide
CGILADPFCLISC

SEQ ID NO: 77 - protein, 13 aa, Artificial
  Synthetic Peptide
CPEANSWVYCSTC

SEQ ID NO: 78 - protein, 13 aa, Artificial
  Synthetic Peptide
CAPTSGWIYCSTC

SEQ ID NO: 79 - protein, 12 aa, Artificial
  Synthetic Peptide
CIPLDWTCMIAC

SEQ ID NO: 80 - protein, 13 aa, Artificial
  Synthetic Peptide
  Xaa (7)..(7): Xaa is Agb
CPYVAGXGTCLLC

SEQ ID NO: 81 - protein, 13 aa, Artificial
  Synthetic Peptide
  Xaa (7)..(7): Xaa is Arg(Me)
CPYVAGXGTCLLC

SEQ ID NO: 82 - protein, 13 aa, Artificial
  Synthetic Peptide
  Xaa (12)..(12): Xaa is Cba
CPYVAGRGTCLXC

SEQ ID NO: 83 - protein, 13 aa, Artificial
  Synthetic Peptide
  Xaa (11)..(11): Xaa is Cba
CPYVAGRGTCXLC

SEQ ID NO: 84 - protein, 13 aa, Artificial
  Synthetic Peptide
CPYVAGRATCLLC

SEQ ID NO: 85 - protein, 13 aa, Artificial
  Synthetic Peptide
  Xaa (7)..(7): Xaa is HArg
CPYVAGXGTCLLC

SEQ ID NO: 86 - protein, 13 aa, Artificial
  Synthetic Peptide
  Xaa (12)..(12): Xaa is tBuAla
CPYVAGRGTCLXC

SEQ ID NO: 87 - protein, 13 aa, Artificial
  Synthetic Peptide
  Xaa (11)..(11): Xaa is tBuAla
CPYVAGRGTCXLC

SEQ ID NO: 88 - protein, 13 aa, Artificial
  Synthetic Peptide
  Xaa (7)..(7): Xaa is Agb
  Xaa (12)..(12): Xaa is tBuAla
CPYVAGXATCLXC

SEQ ID NO: 89 - protein, 13 aa, Artificial
  Synthetic Peptide
  Xaa (10)..(10): Xaa is Agb
CYNHANPVCXYYC

SEQ ID NO: 90 - protein, 13 aa, Artificial
  Synthetic Peptide
  Xaa (10)..(10): Xaa is Agb
CASPDNPVCXFYC

SEQ ID NO: 91 - protein, 13 aa, Artificial
  Synthetic Peptide
  Xaa (10)..(10): Xaa is Arg(Me)
CASPDNPVCXFYC

SEQ ID NO: 92 - protein, 14 aa, Artificial
  Synthetic Peptide
  Xaa (11)..(11): Xaa is HArg
CCASPDNPVCXFYC

SEQ ID NO: 93 - protein, 13 aa, Artificial
  Synthetic Peptide
CANPDNPVCRFYC

SEQ ID NO: 94 - protein, 13 aa, Artificial
  Synthetic Peptide
CRNPDNPVCRFYC

SEQ ID NO: 95 - protein, 13 aa, Artificial
  Synthetic Peptide
CHNPSNPVCRFYC

SEQ ID NO: 96 - protein, 13 aa, Artificial
  Synthetic Peptide
CVNKHNPVCRFYC

SEQ ID NO: 97 - protein, 13 aa, Artificial
  Synthetic Peptide
CVNAENPVCRFYC

SEQ ID NO: 98 - protein, 13 aa, Artificial
  Synthetic Peptide
CQNPGNPVCRFYC

SEQ ID NO: 99 - protein, 13 aa, Artificial
  Synthetic Peptide
CMNPDNPVCRFYC

SEQ ID NO: 100 - protein, 13 aa, Artificial
  Synthetic Peptide
CYNQENPVCRFYC

SEQ ID NO: 101 - protein, 13 aa, Artificial
  Synthetic Peptide
CNNPANPVCRFYC

SEQ ID NO: 102 - protein, 13 aa, Artificial
  Synthetic Peptide
CFNIDNPVCRFYC

SEQ ID NO: 103 - protein, 13 aa, Artificial
  Synthetic Peptide
CSNPENPVCRFYC

SEQ ID NO: 104 - protein, 13 aa, Artificial
  Synthetic Peptide
CMNEDNPVCRFYC

SEQ ID NO: 105 - protein, 13 aa, Artificial
  Synthetic Peptide
CMNEANPVCRFYC

SEQ ID NO: 106 - protein, 13 aa, Artificial
  Synthetic Peptide
CHNLDNPVCRFYC

SEQ ID NO: 107 - protein, 13 aa, Artificial
  Synthetic Peptide
CANHDNPVCRFYC

SEQ ID NO: 108 - protein, 13 aa, Artificial
  Synthetic Peptide
CKNYDNPVCRFYC

SEQ ID NO: 109 - protein, 13 aa, Artificial
  Synthetic Peptide
CENMDNPVCRFYC

SEQ ID NO: 110 - protein, 13 aa, Artificial
  Synthetic Peptide
CMNTDNPVCRFYC

SEQ ID NO: 111 - protein, 13 aa, Artificial
  Synthetic Peptide
CLNVDNPVCRFYC

SEQ ID NO: 112 - protein, 13 aa, Artificial
  Synthetic Peptide
CLNPDNPVCRFYC

SEQ ID NO: 113 - protein, 13 aa, Artificial
  Synthetic Peptide
  Xaa (10)..(10): Xaa is HArg
CYNHANPVCXYYC

SEQ ID NO: 114 - protein, 13 aa, Artificial
  Synthetic Peptide
  Xaa (10)..(10): Xaa is Arg(Me)
CYNHANPVCXYYC

SEQ ID NO: 115 - protein, 13 aa, Artificial
  Synthetic Peptide
  Xaa (7)..(7): Xaa is HArg
  Xaa (12)..(12): Xaa is tBuAla
CPYVAGXATCLXC

SEQ ID NO: 116 - DNA, 26 bp, Artificial
  Synthetic Primer
acaggtacgttaatagttaatagcgt

SEQ ID NO: 117 - DNA, 22 bp, Artificial
  Synthetic Primer
atattgcagcagtacgcacaca

SEQ ID NO: 118 - DNA, 26 bp, Artificial
  Synthetic Primer
acactagccatccttactgcgcttcg